SEQUENCE LISTING (14 sequences)

SEQ ID NO: 1 - protein, 148 aa, Artificial sequence
  LOX  auto-inhibitory LPD amino acid sequence
APPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREPGASRAENQTAPGEVPALSNLRPPSRVDGMVGD

SEQ ID NO: 2 - DNA, 444 bp, Artificial sequence
  LOX  auto-inhibitory LPD nucleic acid sequence
gcgccgccggcggctggtcagcagcagccgccgcgtgaaccaccggcggcaccgggcgcctggcgccagcagatccagtgggaaaacaacggtcaggtgttcagcctgctgtctctgggctctcagtatcagccgcagcgtcgtcgtgatccgggcgcggcagttccgggtgctgcgaacgcttctgcgcagcagccgcgtaccccgattctgctgatccgtgacaatcgtaccgctgcagcccgtacccgtactgcgggcagctccggcgttaccgctggtcgtccgcgtcctaccgctcgtcattggtttcaggctggctactctacgtctcgtgcgcgtgaagcgggcgcatcccgcgctgaaaaccagaccgctccgggtgaagtaccggcgctgagcaacctgcgcccgccgagccgcgtggatggcatggtgggcgac

SEQ ID NO: 3 - protein, 417 aa, Artificial sequence
  Full length LOX amino acid sequence
MRFAWTVLLLGPLQLCALVHCAPPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 4 - DNA, 1251 bp, Artificial sequence
  Full length LOX nucleic acid sequence
atgcgcttcgcctggaccgtgctcctgctcgggcctttgcagctctgcgcgctagtgcactgcgcgccgccggcggctggtcagcagcagccgccgcgtgaaccaccggcggcaccgggcgcctggcgccagcagatccagtgggaaaacaacggtcaggtgttcagcctgctgtctctgggctctcagtatcagccgcagcgtcgtcgtgatccgggcgcggcagttccgggtgctgcgaacgcttctgcgcagcagccgcgtaccccgattctgctgatccgtgacaatcgtaccgctgcagcccgtacccgtactgcgggcagctccggcgttaccgctggtcgtccgcgtcctaccgctcgtcattggtttcaggctggctactctacgtctcgtgcgcgtgaagcgggcgcatcccgcgctgaaaaccagaccgctccgggtgaagtaccggcgctgagcaacctgcgcccgccgagccgcgtggatggcatggtgggcgacgatccatacaatccgtacaaatattctgatgacaacccgtattacaactattacgatacttacgaacgcccacgccctggtggccgttaccgtccgggttacggcaccggttacttccagtacggcctgccggatctggtggcggacccgtattatatccaggcgtccacttacgttcagaaaatgtctatgtacaacctgcgttgcgcggcagaagaaaactgcctggccagtacggcgtatcgcgccgacgtgcgtgactacgaccaccgcgtgttactgcgtttcccacagcgtgttaaaaaccagggtaccagcgatttcctgccgtctcgtccgcgctactcctgggaatggcatagctgccaccagcactatcattccatggacgaattttcccattatgacctgctggatgcaaacacccagcgccgcgtcgcagaaggccacaaagcgtccttttgcctggaagacacctcctgcgattacggctaccaccgccgttttgcgtgcaccgcgcacacccagggcctgtccccgggctgctacgacacctacggcgccgacatcgactgccagtggatcgatatcaccgatgttaaaccgggcaactatatcctgaaagttagcgtgaacccgagctacctggtgccggaaagcgattacaccaacaacgtggttcgttgcgacatccgttacaccggtcaccacgcgtatgcgtctggttgcaccatttctccgtac

SEQ ID NO: 5 - protein, 413 aa, Artificial sequence
  Exemplary amino acid sequence of a LPD fused to an Fc domain
METDTLLLWVLLLWVPGSTGAGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKASGAGSTTENLYFQGGSAPPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVGD

SEQ ID NO: 6 - DNA, 1239 bp, Artificial sequence
  Exemplary nucleic acid sequence of a LPD fused to an Fc domain
atggagacagacacactcctgctatgggtactgctgctctgggttccaggttccactggcgccggatcaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaagctagcggagccggaagcacaaccgaaaacctgtattttcagggcggatccgcgccgccggcggctggtcagcagcagccgccgcgtgaaccaccggcggcaccgggcgcctggcgccagcagatccagtgggaaaacaacggtcaggtgttcagcctgctgtctctgggctctcagtatcagccgcagcgtcgtcgtgatccgggcgcggcagttccgggtgctgcgaacgcttctgcgcagcagccgcgtaccccgattctgctgatccgtgacaatcgtaccgctgcagcccgtacccgtactgcgggcagctccggcgttaccgctggtcgtccgcgtcctaccgctcgtcattggtttcaggctggctactctacgtctcgtgcgcgtgaagcgggcgcatcccgcgctgaaaaccagaccgctccgggtgaagtaccggcgctgagcaacctgcgcccgccgagccgcgtggatggcatggtgggcgac

SEQ ID NO: 7 - protein, 271 aa, Artificial sequence
  Fc tag amino acid sequence
METDTLLLWVLLLWVPGSTGAGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKASGAGSTTENLYFQGGSEFKLDI

SEQ ID NO: 8 - DNA, 795 bp, Artificial sequence
  Fc tag nucleic acid sequence
atggagacagacacactcctgctatgggtactgctgctctgggttccaggttccactggcgccggatcaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaagctagcggagccggaagcacaaccgaaaacctgtattttcagggcggatcc

SEQ ID NO: 9 - protein, 248 aa, Artificial sequence
  LOX catalytic activity domain amino acid sequence
DPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 10 - DNA, 744 bp, Artificial sequence
  LOX catalytic activity domain nucleic acid sequence
gatccatacaatccgtacaaatattctgatgacaacccgtattacaactattacgatacttacgaacgcccacgccctggtggccgttaccgtccgggttacggcaccggttacttccagtacggcctgccggatctggtggcggacccgtattatatccaggcgtccacttacgttcagaaaatgtctatgtacaacctgcgttgcgcggcagaagaaaactgcctggccagtacggcgtatcgcgccgacgtgcgtgactacgaccaccgcgtgttactgcgtttcccacagcgtgttaaaaaccagggtaccagcgatttcctgccgtctcgtccgcgctactcctgggaatggcatagctgccaccagcactatcattccatggacgaattttcccattatgacctgctggatgcaaacacccagcgccgcgtcgcagaaggccacaaagcgtccttttgcctggaagacacctcctgcgattacggctaccaccgccgttttgcgtgcaccgcgcacacccagggcctgtccccgggctgctacgacacctacggcgccgacatcgactgccagtggatcgatatcaccgatgttaaaccgggcaactatatcctgaaagttagcgtgaacccgagctacctggtgccggaaagcgattacaccaacaacgtggttcgttgcgacatccgttacaccggtcaccacgcgtatgcgtctggttgcaccatttctccgtac

SEQ ID NO: 11 - protein, 148 aa, Artificial sequence
  mutated LPD amino acid sequence (N346, 362, 409Q)
APPAAGQQQPPREPPAAPGAWRQQIQWENNGQVFSLLSLGSQYQPQRRRDPGAAVPGAAQASAQQPRTPILLIRDQRTAAARTRTAGSSGVTAGRPRPTARHWFQAGYSTSRAREAGASRAEQQTAPGEVPALSNLRPPSRVDGMVGD

SEQ ID NO: 12 - DNA, 444 bp, Artificial sequence
  mutated LPD nucleic acid sequence (N346, 362, 409Q)
gcgccgccggcggctggtcagcagcagccgccgcgtgaaccaccggcggcaccgggcgcctggcgccagcagatccagtgggaaaacaacggtcaggtgttcagcctgctgtctctgggctctcagtatcagccgcagcgtcgtcgtgatccgggcgcggcagttccgggtgctgcgcaggcttctgcgcagcagccgcgtaccccgattctgctgatccgtgaccagcgtaccgctgcagcccgtacccgtactgcgggcagctccggcgttaccgctggtcgtccgcgtcctaccgctcgtcattggtttcaggctggctactctacgtctcgtgcgcgtgaagcgggcgcatcccgcgctgaacagcagaccgctccgggtgaagtaccggcgctgagcaacctgcgcccgccgagccgcgtggatggcatggtgggcgac

SEQ ID NO: 13 - protein, 413 aa, Artificial sequence
  Fc-mutated LPD amino acid sequence
METDTLLLWVLLLWVPGSTGAGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKASGAGSTTENLYFQGGSAPPAAGQQQPPREPPAAPGAWRQQIQWENNGQVFSLLSLGSQYQPQRRRDPGAAVPGAAQASAQQPRTPILLIRDQRTAAARTRTAGSSGVTAGRPRPTARHWFQAGYSTSRAREAGASRAEQQTAPGEVPALSNLRPPSRVDGMVGD

SEQ ID NO: 14 - DNA, 1239 bp, Artificial sequence
  Fc-mutated LPD nucleic acid sequence
atggagacagacacactcctgctatgggtactgctgctctgggttccaggttccactggcgccggatcaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaagctagcggagccggaagcacaaccgaaaacctgtattttcagggcggatccgcgccgccggcggctggtcagcagcagccgccgcgtgaaccaccggcggcaccgggcgcctggcgccagcagatccagtgggaaaacaacggtcaggtgttcagcctgctgtctctgggctctcagtatcagccgcagcgtcgtcgtgatccgggcgcggcagttccgggtgctgcgcaggcttctgcgcagcagccgcgtaccccgattctgctgatccgtgaccagcgtaccgctgcagcccgtacccgtactgcgggcagctccggcgttaccgctggtcgtccgcgtcctaccgctcgtcattggtttcaggctggctactctacgtctcgtgcgcgtgaagcgggcgcatcccgcgctgaacagcagaccgctccgggtgaagtaccggcgctgagcaacctgcgcccgccgagccgcgtggatggcatggtgggcgac

(19) United States
(12) Patent Application Publication
(10) Pub. No.: US 2022/0049236 A1
(43) Pub. Date: Feb. 17, 2022

(54) COMPOSITIONS COMPRISING THE PROPEPTIDE OF LYSYL OXIDASE AND USES THEREOF

(71) Applicant: Yeda Research and Development Co. Ltd., Rehovot (IL)

(72) Inventors: Irit SAGI, Rehovot (IL); Nikolaos A. AFRATIS, Rehovot (IL)

(73) Assignee: Yeda Research and Development Co. Ltd., Rehovot (IL)

(21) Appl. No.: 17/515,591

(22) Filed: Nov. 1, 2021

Related U.S. Application Data
(63) Continuation of application No. PCT/IL2020/050486, filed on Apr. 30, 2020.
(60) Provisional application No. 62/977,792, filed on Feb. 18, 2020.

(30) Foreign Application Priority Data
May 2, 2019 (IL) 266433

(51) Int. Cl. C12N 9/96; C12N 9/06; A61P 19/04; A61P 21/00; C07K 14/765; C07K 14/79; C07K 14/47

(52) U.S. Cl. CPC C12N 9/96; C12N 9/0022; C12Y 104/03013; A61P 19/04; A61P 21/00; A61K 38/00; C07K 14/79; C07K 14/4708; C07K 2319/30; C07K 2319/31; C07K 14/765

(57) ABSTRACT

A method of treating fibrosis in a subject in need thereof is provided. The method comprising administering to the subject a polypeptide comprising a propeptide of lysyl oxidase (LOX), the polypeptide being devoid of LOX catalytic activity, thereby treating the fibrosis in the subject, wherein the method does not comprise administration of D-penicillamine. Also provided are polypeptide compositions for use in therapy.

### RELATED APPLICATIONS

This application is a Continuation of PCT Patent Application No. PCT/IL2020/050486 having International filing date of Apr. 30, 2020, which claims the benefit of priority of U.S. Provisional Patent Application No. 62/977,792 filed on Feb. 18, 2020 and Israel Patent Application No. 266433 filed on May 2, 2019. The contents of the above applications are all incorporated by reference as if fully set forth herein in their entirety.

### SEQUENCE LISTING STATEMENT

The ASCII file, entitled 89718SequenceListing.txt, created on Nov. 1, 2021, comprising 28,106 bytes, submitted concurrently with the filing of this application is incorporated herein by reference.

### FIELD AND BACKGROUND OF THE INVENTION

The present invention, in some embodiments thereof, relates to compositions comprising the propeptide of Lysyl Oxidase (LOX) and uses thereof.

DMD is the most common and severe childhood form of a family of muscular dystrophies caused by a mutation in the X-linked dystrophin gene. Consequently, the anchorage of the myofiber to the ECM is lost and repeated muscle contraction leads to mechanical breakdown of muscle fibers. DMD causes progressive weakness and loss (atrophy) of skeletal and heart muscle [1]. Early signs of DMD may include delayed ability to sit, stand, or walk and difficulties learning to speak. Muscle weakness is usually noticeable by 3 or 4 years of age and begins in the hips, pelvic area, upper legs, and shoulders. Damaged fibers induce satellite cells (SC) activation and muscle regeneration [2]. Smooth muscles, such as those aligning the GI tract, are also significantly affected in the disease and patients often suffer from intestinal paresis [3].

The ‘muscle regeneration program’ is a multistep process. Initially, a transient inflammatory response occurs, during which growth factors and cytokines are released to the injured area to allow the proliferation and recruitment of macrophages and fibroblasts that dispose muscle debris and lay down ECM. Formation of myofibers begins with activation of SC followed by proliferation, differentiation and fusion to new or present myocytes [2,4]. TGFβ1, a potent regulator of tissue wound healing and fibrosis, is upregulated in skeletal muscles following injury and presumably participates in the transient inflammatory response. However, in dystrophic diseases such as DMD, as a consequence of the constant muscle injuries, a persistent inflammatory response takes place. High levels of proinflammatory cytokines, growth factors, nitric oxide and TGFβ that are produced by inflammatory cells create a stressful environment affecting the balance between SC and fibroblasts [5]. As a result, a shift in the balance between connective (ECM and fibroblasts) and muscle tissue occurs. Excess components of the ECM, such as collagen and fibrinogen, are deposited at the expense of muscle forming a highly fibrotic tissue [5]. Remarkably, such ECM dysregulated processes lead to many of the pathological features of DMD which are not directly caused only by the lack of Dystrophin, but are also due to the complex interactions of the myofibers and SC with the ECM. Thus, the fibrotic muscle delays muscle repair and regeneration, enhances inflammation and exacerbates disease progression. Altogether, loss of regeneration capacity and fibrosis are the major causes for muscle dysfunction as well as of the lethal phenotype of DMD and other related muscular dystrophies [6].

Gene editing and cell transplantation have been suggested as novel approaches for treating DMD. However, these promising approaches either rely on an early age of intervention, before waves of necrosis lead to muscle fiber loss, replacement of tissue by fat and fibrosis, or on a multimodal therapy that will tackle the fibrotic reaction [7,8]. Moreover, major side effects, such as balance problems and vomiting are associated with these treatment modalities. Although these approaches increase dystrophin production, which would predict improvement in muscle function, this has not yet been shown. Therefore, identification of genes associated with the physical tissue damage, as well as the development of treatment to cope with undesired ECM remodeling outline an unmet need in DMD.

In an attempt to identify genes underlying the lethal phenotype of DMD, large scale gene expression analyses derived from numerous human DMD patients and mdx mice were carried out. Genes significantly up- or down-regulated in the murine model system and human patients were considered as DMD core genes. One of the genes identified in these analyses to be highly over-expressed in DMD is LOX. Its elevated expression was further confirmed using western blot and immunohistochemistry analyses also in golden retriever muscular dystrophy (GRMD), the canine model for DMD [9,10].

LOX was primarily identified as an extracellular enzyme which initiates the covalent cross-linking of collagen and elastin molecules. LOX is the most abundant family member of its family [LOX, LOX-Like (LOXL) 1-4] that initiates these post-translational modifications. Its activity is critical for maintaining the tensile and elastic features of connective tissues in the skeletal, pulmonary and cardiovascular systems [11-14]. LOX is synthesized and secreted as a 50-kDa inactive proenzyme, which is processed by proteolytic cleavage of 18-kDa prodomain by BMP-1 to a functional 32-kDa enzyme (LOX). Accordingly, Lox mutant mice (Lox^{-/-}) die upon birth due to aortic aneurysms and rupturing of their diaphragms [15-17] caused by matrix organization defects [18]. Recent work has directly linked LOX activity to a number of human pathologies including fibrosis, neurodegeneration and cancer (e.g., [12,13,11]). Accumulating evidence suggests that apart from their classical extracellular activities, members of the LOX family are also found intracellularly within the cytoplasm and the nucleus (e.g., [19-21]). These intracellular LOX activities have been associated with regulation of cellular motility and migration, gene transcription, and differentiation [12,22-26]. Altogether, results demonstrate that LOX plays multiple independent roles—both within cells and in the ECM. Recently, it was shown that Lox mutant mice display severe skeletal muscle defects [27]. In these mice, the amount of myofibers is significantly reduced, yet they display an excessive deposition of ECM and fibroblasts. Moreover, these Lox-muscle related phenotypes are rescued upon the inhibition of TGFβ signaling thus demonstrating that a Lox-TGFβ feedback loop regulates the balance between the amounts of muscle versus those of connective tissue.

Additional background art includes:
Thomassin et al. J Biol Chem. 2005 Dec. 30; 280(52):42848-55. Epub 2005 Oct. 26.
Grimsby et al. J Cell Biochem. Author manuscript; available in PMC 2013 Dec. 11.
Zhao et al. J Biol Chem. 2009 Jan. 16; 284(3): 1385-1393.
U.S. Publication No. 20080261870
U.S. Publication No. 20170360732.

### SUMMARY OF THE INVENTION

According to an aspect of some embodiments of the present invention there is provided a synthetic polypeptide comprising a propeptide of human lysyl oxidase (LOX), the polypeptide being devoid of LOX catalytic activity, the synthetic polypeptide comprising a modification which imparts the polypeptide with enhanced stability under physiological conditions as compared to a native form of the polypeptide not comprising the modification.

According to some embodiments of the invention, the modification comprises a proteinaceous modification.

According to some embodiments of the invention, the proteinaceous modification is selected from the group consisting of immunoglobulin, human serum albumin, and transferrin.

According to some embodiments of the invention, the immunoglobulin comprises an Fc domain.

According to some embodiments of the invention, the polypeptide is a chimeric polypeptide.

According to some embodiments of the invention, the modification comprises a chemical modification.

According to some embodiments of the invention, the chemical modification is a polymer.

According to some embodiments of the invention, the polymer is selected from the group consisting of a polycationic polymer, a non-ionic water-soluble polymer, a polyether polymer and a biocompatible polymer.

According to an aspect of some embodiments of the present invention there is provided a method of treating fibrosis in a subject in need thereof, the method comprising administering to the subject a polypeptide comprising a propeptide of lysyl oxidase (LOX), the polypeptide being devoid of LOX catalytic activity, thereby treating the fibrosis in the subject, wherein the method does not comprise administration of D-penicillamine.

According to an aspect of some embodiments of the present invention there is provided a method of treating Duchenne Muscular Dystrophy (DMD) in a subject in need thereof, the method comprising administering to the subject a polypeptide comprising a propeptide of lysyl oxidase (LOX), the polypeptide being devoid of LOX catalytic activity, thereby treating the DMD in the subject.

According to some embodiments of the invention, the polypeptide is glycosylated on at least one of N81, N97 and N144 of SEQ ID NO: 1.

According to some embodiments of the invention, the polypeptide is glycosylated on at least two of N81, N97 and N144 of SEQ ID NO: 1.

According to some embodiments of the invention, the polypeptide is glycosylated on N81, N97 and N144 of SEQ ID NO: 1.

According to some embodiments of the invention, the polypeptide is not glycosylated on N81, N97 and N144 of SEQ ID NO: 1.

According to some embodiments of the invention, the polypeptide is characterized by an EC₅₀ of 10-500 nM, as determined by an ELISA assay.

According to some embodiments of the invention, the polypeptide is characterized by KD of 10-100 nM, as determined by a microscale thermophoresis.

According to some embodiments of the invention, the polypeptide is characterized by a transition midpoint of 20-70° C., as determined by differential scanning fluorimetry (DSF).

According to some embodiments of the invention, the polypeptide is capable of reducing at least one of fibrillar collagen and cross-linked collagen, as determined by SHG microscopy. According to some embodiments of the invention, the polypeptide is capable of altering collagen fiber orientation, as determined by SHG microscopy.

According to some embodiments of the invention, the fibrosis is not Ras-signaling dependent.

According to some embodiments of the invention, the fibrosis is not associated with cancer or a bone disease

According to some embodiments of the invention, the propeptide of lysyl oxidase (LOX) is of human LOX.

According to an aspect of some embodiments of the present invention there is provided a method of inhibiting enzymatic activity of LOX, the method comprising contacting the LOX with the polypeptide the polypeptide as described herein, thereby inhibiting the enzymatic activity of LOX.

According to some embodiments of the invention, the method is performed in-vivo.

According to some embodiments of the invention, the method is performed in-vitro.

According to some embodiments of the invention, the method is performed ex-vivo.

According to an aspect of some embodiments of the present invention there is provided a method of producing the polypeptide the polypeptide as described herein, the method comprising expressing in a host cell, a polynucleotide encoding the polypeptide and when necessary, modifying the polypeptide with the chemical modification, thereby producing the polypeptide.

According to some embodiments of the invention, the method further comprises isolating the polypeptide from the host cell.

According to some embodiments of the invention, the polypeptide is as set forth in SEQ ID NO: 1 or 5.

According to an aspect of some embodiments of the present invention there is provided a polynucleotide encoding the polypeptide as described herein.

According to a specific embodiment, the use does not comprise D-penicillamine.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G show the design and biochemical properties of the fusion protein Fc-LPD. FIGS. 1A, 1B, design and generation of the antibody-like LOX inhibitor, Fc-LPD. FIG. 1C, binding affinity of Fc-LPD & Fc-LPD N346, 362, 409Q to LOX catalytic domain, EC₅₀. FIGS. 1D, 1E, measurement of transition midpoint of Fc-LPD monitoring the intrinsic fluorescence signal of Fc-LPD as a measure of its folding state, K_{D}. FIGS. 1F, 1G, a ratiometric measurement of the fluorescent signal of Fc-LPD is plotted against increasing temperature of a chemical denaturant to determine the Tₘ of a protein.

FIGS. 2A-E show interference of ECM assembly, as determined by SHG microscopy. FIG. 2A, scheme illustrating the experimental setup for monitoring different stages in collagen assembly secreted by HDF cells using SHG microscopy, FIG. 2B, representative SHG images of Fc-LPD that interferes with ECM alignment. Representative fiber directionality analysis on each SHG image depicting the frequency of fibers in a specific orientation FIG. 2C, representative SHG images of Fc-LPD & Fc-LPD N346, 362, 409Q that interferes with ECM alignment compare to Fc. FIGS. 2D, 2E, mean entropy and thickness of ECM graphs.

FIGS. 3A-E show the interactions of different Fc-LPD glycosylation forms with ECM proteins released from HDF cells. FIG. 3A, semi-separation of 45 kDa Fc-LPD and 60 kDa Fc-LPD. FIG. 3B, immunoprecipitation of 45 kDa Fc-LPD and 60 kDa Fc-LPD. FIG. 3C, interaction of LOX catalytic domain with Fc-LPD after immunoprecipitation (I.P). FIG. 3D, binding affinity of Fc-LPD & Fc-LPD N346, 362, 409Q against HSP70, EC₅₀. FIG. 3E, binding of HSP70 to LPD by using yeast surface display.

FIGS. 4A-H show early stage of in vivo evaluation of DMD diaphragm and gastrocnemius fibrosis after treatment with Fc-LPD & Fc-LPD N346, 362, 409Q. FIG. 4A, schematic representation of the experiment. FIG. 4B, show the results of the rotarod running assay. FIG. 4C, show H&E and sirius red of mice diaphragm. FIG. 4E, quantification of sirius red of mice diaphragm. FIG. 4F, 4G, show H&E and sirius red of mice gastrocnemius. FIG. 4H, quantification of sirius red of mice gastrocnemius.

FIGS. 5A-I show early stage in vivo evaluation of DMD quadricep and tibialis anterior fibrosis after treatment with Fc-LPD & Fc-LPD N346, 362, 409Q. FIG. 5A, B, show H&E and sirius red representative images in quadricep of DMD mice after Fc, Fc-LPD & Fc-LPD N346, 362, 409Q treatment. FIG. 5C, quantification of sirius red of mice quadricep. FIGS. 5D-F, show two-photon SHG representative images from mice quadricep tissue and quadricep endomysium, respectively. FIG. 5E, 5G quantification of two-photon SHG images of mice quadricep. FIG. 5H, 5I, show two-photon SHG representative images from mice tibialis anterior and quantification of two-photon SHG images of mice tibialis anterior, respectively.

FIGS. 6A-H. show in vivo evaluation of DMD muscle fibrosis after treatment with Fc-LPD. FIG. 6A, Schematic representation of the experiment. FIG. 6B, show the results of the Rotarod running assay, FIG. 6C show the results of the Hanging test assay. FIG. 6D, show H&E, Sirius red and SHG representative images from mice tibialis anterior. FIGS. 6E, 6F, quantification of Sirius red and fibers per area from mice tibialis anterior (TA). FIG. 6G, show semi-qualitative histological evaluation analysis for tibialis anterior tissues. FIG. 6H show representative fiber directionality analysis on each SHG image from mice TA.

FIGS. 7A-E show in vivo evaluation of DMD muscle fibrosis after treatment with Fc-LPD in diaphragm and heart. FIGS. 7A, 7D show H&E and Sirius red representative images in diaphragm and heart of DMD mice after Fc and Fc-LPD treatment, respectively. FIG. 7B, show semi-qualitative histological evaluation for diaphragm tissues. FIGS. 7C and 7E show quantification of diaphragm and heart Sirius red staining.

### DESCRIPTION OF SPECIFIC EMBODIMENTS OF THE INVENTION

The present invention, in some embodiments thereof, relates to compositions comprising the propeptide of Lysyl Oxidase (LOX) and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Embodiments of the invention relate to the development of a protein inhibitor which leverages the levels of extracellular levels of endogenous LOX. With the use of protein engineering a stable form of lysyl oxidase prodomain fused to Fc antibody fragment (Fc-LPD), which specifically inhibits LOX in vitro and in vivo, was generated. Fc-LPD affinity and stability measurements were performed against LOX, determining the binding affinity (EC₅₀=43 nM) and dissociation constant (Kd=32 nM). In vivo studies in mdx mice showed improvement in functional tests, such as rotarod running and hanging test, as well as inhibition of fibrosis accumulation and promotion of normal collagen organization. The present results, demonstrate that inhibition of LOX by Fc-LPD is highly specific and efficiently minimizes the fibrosis and side effects of DMD.

Thus, according to an aspect of the invention there is provided a method of treating fibrosis in a subject in need thereof, the method comprising administering to the subject a polypeptide comprising a propeptide of lysyl oxidase (LOX), the polypeptide being devoid of LOX catalytic activity, thereby treating the fibrosis in the subject, wherein the method does not comprise administration of D-penicillamine.

According to another aspect there is provided a polypeptide comprising a propeptide of lysyl oxidase (LOX) for use in treating fibrosis in a subject in need thereof, the polypeptide being devoid of LOX catalytic activity, wherein the use does not comprise administration of D-penicillamine.

According to another aspect of the invention there is provided a method of treating Duchenne Muscular Dystrophy (DMD) in a subject in need thereof, the method comprising administering to the subject a polypeptide comprising a propeptide of lysyl oxidase (LOX), the polypeptide being devoid of LOX catalytic activity, thereby treating the DMD in the subject.

As used herein “Fibrosis” refers to the accumulation of extracellular matrix constituents that occurs following trauma, inflammation, tissue repair, immunological reactions, cellular hyperplasia, and neoplasia. Examples of tissue fibrosis include, but are not limited to, pulmonary fibrosis, renal fibrosis, cardiac fibrosis, cirrhosis and fibrosis of the liver, skin scars and keloids, adhesions, fibromatosis, atherosclerosis, and amyloidosis.

In some embodiments, the fibrotic condition is primary fibrosis. In some embodiments, the fibrotic condition is idiopathic. In some embodiments, the fibrotic condition is associated with (e.g., is secondary to) a disease (e.g., an infectious disease, an inflammatory disease, an autoimmune disease, a malignant or cancerous disease, and/or a connective disease); a toxin; an insult (e.g., an environmental hazard (e.g., asbestos, coal dust, polycyclic aromatic hydrocarbons), cigarette smoking, a wound); a medical treatment (e.g., surgical incision, chemotherapy or radiation), or a combination thereof.

In some embodiments, the fibrotic condition is a fibrotic condition of the lung, a fibrotic condition of the liver, a fibrotic condition of the heart or vasculature, a fibrotic condition of the kidney, a fibrotic condition of the skin, a fibrotic condition of the gastrointestinal tract, a fibrotic condition of the bone marrow or a hematopoietic tissue, a fibrotic condition of the nervous system, or a combination thereof. In some embodiments, the fibrotic condition affects a tissue chosen from one or more of muscle, tendon, cartilage, skin (e.g., skin epidermis or endodermis), cardiac tissue, vascular tissue (e.g., artery, vein), pancreatic tissue, lung tissue, liver tissue, kidney tissue, uterine tissue, ovarian tissue, neural tissue, testicular tissue, peritoneal tissue, colon, small intestine, biliary tract, gut, bone marrow, or hematopoietic tissue.

In some embodiments, the fibrotic condition is a fibrotic condition of the lung. In some embodiments, the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), bronchiolitis obliterans, or bronchiectasis. In some embodiments, the fibrosis of the lung is secondary to a disease, a toxin, an insult, a medical treatment, or a combination thereof. In some embodiments, fibrosis of the lung is associated with one or more of: a disease process such as asbestosis and silicosis; an occupational hazard; an environmental pollutant; cigarette smoking; an autoimmune connective tissue disorders (e.g., rheumatoid arthritis, scleroderma and systemic lupus erythematosus (SLE)); a connective tissue disorder such as sarcoidosis; an infectious disease, e.g., infection, particularly chronic infection; a medical treatment, including but not limited to, radiation therapy, and drug therapy, e.g., chemotherapy (e.g., treatment with as bleomycin, methotrexate, amiodarone, busulfan, and/or nitrofurantoin). In some embodiments, the fibrotic condition of the lung treated with the methods of the invention is associated with (e.g., secondary to) a cancer treatment, e.g., treatment of a cancer (e.g. squamous cell carcinoma, testicular cancer, Hodgkin's disease with bleomycin). In some embodiments, the fibrotic condition is a fibrotic condition of the liver. In certain embodiments, the fibrotic condition of the liver is chosen from one or more of: fatty liver disease, steatosis (e.g., nonalcoholic steatohepatitis (NASH), cholestatic liver disease (e.g., primary biliary cirrhosis (PBC), cirrhosis, alcohol-induced liver fibrosis, biliary duct injury, biliary fibrosis, cholestasis or cholangiopathies. In some embodiments, hepatic or liver fibrosis includes, but is not limited to, hepatic fibrosis associated with alcoholism, viral infection, e.g., hepatitis (e.g., hepatitis C, B or D), autoimmune hepatitis, non-alcoholic fatty liver disease (NAFLD), progressive massive fibrosis, exposure to toxins or irritants (e.g., alcohol, pharmaceutical drugs and environmental toxins).

In some embodiments, the fibrotic condition is a fibrotic condition of the heart. In certain embodiments, the fibrotic condition of the heart is myocardial fibrosis (e.g., myocardial fibrosis associated with radiation myocarditis, a surgical procedure complication (e.g., myocardial post-operative fibrosis), infectious diseases (e.g., Chagas disease, bacterial, trichinosis or fungal myocarditis)); granulomatous, metabolic storage disorders (e.g., cardiomyopathy, hemochromatosis); developmental disorders (e.g., endocardial fibroelastosis); arteriosclerotic, or exposure to toxins or irritants (e.g., drug induced cardiomyopathy, drug induced cardiotoxicity, alcoholic cardiomyopathy, cobalt poisoning or exposure). In some embodiments, the myocardial fibrosis is associated with an inflammatory disorder of cardiac tissue (e.g., myocardial sarcoidosis).

In some embodiments, the fibrotic condition is a fibrotic condition of the kidney. In some embodiments, the fibrotic condition of the kidney is chosen from one or more of: renal fibrosis (e.g., chronic kidney fibrosis), nephropathies associated with injury/fibrosis (e.g., chronic nephropathies associated with diabetes (e.g., diabetic nephropathy)), lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, HIV-associated nephropathy, or fibrosis associated with exposure to a toxin, an irritant, or a chemotherapeutic agent.

In some embodiments, the fibrotic condition is a fibrotic condition of the skin. In some embodiments, the fibrotic condition of the skin is chosen from one or more of: skin fibrosis, scleroderma, nephrogenic systemic fibrosis (e.g., resulting after exposure to gadolinium which is frequently used as a contrast substance for MRIs in patients with severe kidney failure), scarring and keloid.

In some embodiments, the fibrotic condition is a fibrotic condition of the gastrointestinal tract. In some embodiments, the fibrotic condition is chosen from one or more of fibrosis associated with scleroderma; radiation induced gut fibrosis; fibrosis associated with a foregut inflammatory disorder such as Barrett's esophagus and chronic gastritis, and/or fibrosis associated with a hindgut inflammatory disorder, such as inflammatory bowel disease (IBD), ulcerative colitis and Crohn's disease.

In some embodiments, the fibrotic condition is adhesions. In some embodiments, the adhesions are chosen from one or more of: abdominal adhesions, peritoneal adhesions, pelvic adhesions, pericardial adhesions, peridural adhesions, peritendinous or adhesive capsulitis.

In some embodiments, the fibrotic condition is a fibrotic condition of the eye. In some embodiments, the fibrotic condition of the eye involves diseases of the anterior segment of the eye such as glaucoma and corneal opacification; in some embodiments, the fibrotic condition of the eye involves disease of the posterior segment of the eye such as age-related macular degeneration, diabetic retinopathy, retinopathy of prematurity and neovascular glaucoma; in some embodiments, the fibrotic condition of the eye results from fibrosis following ocular surgery.

In some embodiments, the fibrotic condition is a fibrotic condition of the bone marrow or a hematopoietic tissue. In some embodiments, the fibrotic condition of the bone marrow is an intrinsic feature of a chronic myeloproliferative neoplasm of the bone marrow, such as primary myelofibrosis (also referred to herein as angiogenic myeloid metaplasia or chronic idiopathic myelofibrosis). In some embodiments, the bone marrow fibrosis is associated with (e.g., is secondary to) a malignant condition or a condition caused by a clonal proliferative disease. In some embodiments, the bone marrow fibrosis is associated with a hematologic disorder (e.g., a hematologic disorder chosen from one or more of polycythemia vera, essential thrombocythemia, myelodysplasia, hairy cell leukemia, lymphoma (e.g., Hodgkin or non-Hodgkin lymphoma), multiple myeloma or chronic myelogeneous leukemia (CIVIL)). In some embodiments, the bone marrow fibrosis is associated with (e.g., secondary to) a non-hematologic disorder (e.g., a non-hematologic disorder chosen from solid tumor metastasis to bone marrow, an autoimmune disorder (e.g., systemic lupus erythematosus, scleroderma, mixed connective tissue disorder, or polymyositis), an infection (e.g., tuberculosis), or secondary hyperparathyroidism associated with vitamin D deficiency.

In some embodiments, the fibrosis is not associated with Ras signaling for cell transformation. Examples of such conditions include, cancer e.g., colon, breast, lung or prostate cancer, disorders of the kidney, cardiovascular system and immune system, bone disease, e.g., osteopenic condition, e.g., osteoporosis.

According to a specific embodiment, the fibrotic condition is of the muscle.

There are two major types of muscle tissue in an animal-striated muscle and smooth muscle.

Muscles may also be grouped by location or function. In some embodiments, the polypeptide may be targeted to a tissue of interest e.g., one or more muscles of the face, one or more muscles for mastication, one or more muscles of the tongue and neck, one or more muscles of the thorax, one or more muscles of the pectoral girdle and arms, one or more muscles of the arm and shoulder, one or more ventral and dorsal forearm muscles, one or more muscles of the hand, one or more muscles of the erector spinae, one or more muscles of the pelvic girdle and legs, and/or one or more muscles of the foreleg and foot.

In some embodiments, muscles of the face include, but are not limited to, intraocular muscles such as ciliary, iris dilator, iris sphincter, muscles of the ear such as auriculares, temporoparietalis, stapedius, tensor tympani; muscles of the nose such as procerus, nasalis, dilator naris, depressor septi nasi, levator labii superioris alaeque nasi; muscles of the mouth such as levator anguli oris, depressor anguli oris, orbicularis oris, Buccinator, Zygomaticus Major and Minor, Platysma, Levator Labii Superioris, Depressor Labii Inferioris, Risorius, Mentalis, and/or Corrugator Supercilii.

In some embodiments, muscles of mastication include, but are not limited to, Masseter, Temporalis, Medial Pterygoid, Lateral Pterygoid. In some embodiments, muscles of the tongue and neck include, but are not limited to, Genioglossus, Styloglossus, Palatoglossus, Hyoglossus, Digastric, Stylohyoid, Mylohyoid, Geniohyoid, Omohyoid, Sternohyoid, Sternothyroid, Thyrohyoid, Sternocleidomastoid, Anterior Scalene, Middle Scalene, and/or Posterior Scalene.

In some embodiments, muscles of the thorax, pectoral girdle, and arms include, but are not limited to, Subclavius Pectoralis major, Pectoralis minor, Rectus abdominis, External abdominal oblique, Internal abdominal oblique, Transversus Abdominis, Diaphragm, External Intercostals, Internal Intercostals, Serratus Anterior, Trapezius, Levator Scapulae, Rhomboideus Major, Rhomboideus Minor, Latissimus dorsi, Deltoid, subscapularis, supraspinatus, infraspinatus, Teres major, Teres minor, and/or Coracobrachialis.

In some embodiments, muscles of the arm and shoulder include, but are not limited to, Biceps brachii-Long Head, Biceps brachii-Short Head, Triceps brachii-Long Head, Triceps brachii Lateral Head, Triceps brachii-Medial Head, Anconeus, Pronator teres, Supinator, and/or Brachialis.

In some embodiments, muscles of the ventral and dorsal forearm include, but are not limited to, Brachioradialis, Flexor carpi radialis, Flexor carpi ulnaris, Palmaris longus, Extensor carpi ulnaris, Extensor carpi radialis longus, Extensor carpi radialis brevis, Extensor digitorum, Extensor digiti minimi.

In some embodiments, muscles of the hand include, but are not limited to intrinsic muscles of the hand such as thenar, abductor pollicis brevis, flexor pollicis brevis, opponens pollicis, hypothenar, abductor digiti minimi, the flexor digiti minimi brevis, opponens digiti minimi, palmar interossei, dorsal interossei and/or lumbricals.

In some embodiments, muscles of the erector spinae include, but are not limited to, cervicalis, spinalis, longis simus, and/or iliocostalis.

In some embodiments, muscles of the pelvic girdle and the legs include, but are not limited to, Psoas Major, Iliacus, quadratus femoris, Adductor longus, Adductor brevis, Adductor magnus, Gracilis, Sartorius, Quadriceps femoris such as, rectus femoris, vastus lateralis, vastus medialis, vastus intermedius, Gastrocnemius, Fibularis (Peroneus) Longus, Soleus, Gluteus maximus, Gluteus medius, Gluteus minimus, Hamstrings: Biceps Femoris: Long Head, Hamstrings: Biceps Femoris: Short Head, Hamstrings: Semitendinosus, Hamstrings: Semimembranosus, Tensor fasciae latae, Pectineus, and/or Tibialis anterior.

In some embodiments, muscles of the foreleg and foot include, but are not limited to, Extensor digitorum longus, Extensor hallucis longus, peroneus brevis, plantaris, Tibialis posterior, Flexor hallucis longus, extensor digitorum brevis, extensor hallucis brevis, Abductor hallucis, flexor hallucis brevis, Abductor digiti minimi, flexor digiti minimi, opponens digiti minimi, extensor digitorum brevis, lumbricales of the foot, Quadratus plantae or flexor accessorius, flexor digitorum brevis, dorsal interossei, and/or plantar interossei.

Muscular dystrophies are a group of inherited disorders that cause degeneration of muscle, leading to weak and impaired movements. A central feature of all muscular dystrophies is that they are progressive in nature. Muscular dystrophies include, but are not limited to: Duchenne muscular dystrophy (DMD), Becker muscular dystrophy, Emery-Dreifuss muscular dystrophy, facioscapulohumeral muscular dystrophy, limb-girdle muscular dystrophies, and myotonic dystrophy Types 1 and 2, including the congenital form of Myotonic dystrophy Type 1. Symptoms may vary by type of muscular dystrophy with some or all muscles being affected.

Exemplary symptoms of muscular dystrophies include delayed development of muscle motor skills, difficulty using one or more muscle groups, difficulty swallowing, speaking or eating, drooling, eyelid drooping, frequent falling, loss of strength in a muscle or group of muscles as an adult, loss in muscle size, problems walking due to weakness or altered biomechanics of the body, muscle hypertrophy, muscle pseudohypertrophy, fatty infiltration of muscle, replacement of muscle with non-contractile tissue (e.g., muscle fibrosis), muscle necrosis, and/or cognitive or behavioral impairment/mental retardation.

While there are no known cures for muscular dystrophics, several supportive treatments are used which include both symptomatic and disease modifying therapies. Corticosteroids, physical therapy, orthotic devices, wheelchairs, or other assistive medical devices for ADLs and pulmonary function are commonly used in muscular dystrophies. Cardiac pacemakers are used to prevent sudden death from cardiac arrhythmias in Myotonic dystrophy. Anti-myotonic agents which improve the symptoms of myotonia (inability to relax) include mexilitine, and in some cases phenytoin, procainamide and quinine. These can be combined with the treatment with the polypeptide of some embodiments of the invention.

Duchenne muscular dystrophy (DMD) is characterized by weakness in the proximal muscles, abnormal gait, pseudohypertrophy in the gastrocnemius (calf) muscles, and elevated creatine kinase (CK). Many DMD patients are diagnosed around the age of 5, when symptoms/signs typically become more obvious. Affected individuals typically stop walking around age 10-13 and die in or before their mid to late 20's due to cardiorespiratory dysfunction.

The disorder DMD is caused by a mutation in the dystrophin gene, located on the human X chromosome, which codes for the protein dystrophin, an important structural component within muscle tissue that provides structural stability to the dystroglycan complex (DGC) of the cell membrane. Dystrophin links the internal cytoplasmic actin filament network and extracellular matrix, providing physical strength to muscle fibers. Accordingly, alteration or absence of dystrophin results in abnormal sarcolemma membrane tearing and necrosis of muscle fibers. While persons of both sexes can carry the mutation, females rarely exhibit severe signs of the disease.

A main symptom of DMD is muscle weakness associated with muscle wasting with the voluntary muscles being first affected typically, especially affecting the muscles of the hips, pelvic area, thighs, shoulders, and calf muscles. Muscle weakness also occurs in the arms, neck, and other areas. Calves are often enlarged. Signs and symptoms usually appear before age 6 and may appear as early as infancy. Other physical symptoms include, but are not limited to, delayed ability to walk independently, progressive difficulty in walking, stepping, or running, and eventual loss of ability to walk (usually by the age of 15); frequent falls; fatigue; difficulty with motor skills (running, hopping, jumping); increased lumbar lordosis, leading to shortening of the hip-flexor muscles; contractures of Achilles tendon and hamstrings impairing functionality because the muscle fibers shorten and fibrosis occurs in connective tissue; muscle fiber deformities; pseudohypertrophy (enlargement) of tongue and calf muscles caused by replacement of muscle tissue by fat and connective tissue; higher risk of neurobehavioral disorders (e.g., ADHD), learning disorders (dyslexia), and non-progressive weaknesses in specific cognitive skills (in particular short-term verbal memory); skeletal deformities (including scoliosis in some cases).

As used herein “treating” refers to abrogating, substantially inhibiting, slowing or reversing the progression of a condition, substantially ameliorating clinical or aesthetical symptoms of a condition or substantially preventing the appearance of clinical or aesthetical symptoms of a condition.

As used herein “subject” refers to a subject diagnosed with or at risk of fibrosis.

As used herein “synthetic polypeptide” refers to a protein not occurring in nature, either because it is isolated from a natural environment thereof e.g., the human or animal body, or because it is mutated with respect to the wild-type form or because it is modified e.g., attached to a heterologous moiety e.g., protein or chemical.

As used herein “Lysyl Oxidase” or “LOX” refers to the protein product of the LOX gene. In humans, the LOX gene is located on chromosome 5q23.3-31.2. The primary sequence of the LOX protein is highly conserved in mammals. Human LOX is synthesized as a pre-proprotein (pre-pro-LOX) of 417 amino acids (UniProtKB-P28300, which undergoes a number of post-translational modifications within the endoplasmic reticulum (ER) and post-ER e.g., glycosylation as described below. After cleavage of the 21 amino acid signal sequence, the N-terminal propeptide, comprising 147 amino acid residues, is N-glycosylated and the C-terminal sequence containing the 249 amino acid residue mature protein which is also referred to herein as the part which comprises the LOX catalytic activity, is distinctively folded to acquire at least three disulfide bonds. Copper is a cofactor of the functional catalyst, incorporated into the nascent enzyme within the ER. The enzyme also contains a peptidyl organic cofactor, lysyltyrosine quinone (LTQ) generated by an intramolecular cross-link between lysine 320 and the copper-dependent oxidation product of tyrosine 355.

The “LOX-propeptide” (LOX-PP) or “LOX-prodomain” (LPD) is the N-terminal propeptide corresponding to amino acid residues 22-168 of SEQ ID NO: 3, which following secretion of proLOX to the extracellular space, is cleaved by procollagen-C-proteinase (BMP-1) or BMP-1-related metalloproteinases, to generate the free propeptide (LPD) and the catalytically active LOX.

A key intracellular function of LOX-PP (LPD) is most likely the maintenance of lysyl oxidase in an inactive state within the secretory pathway. Propeptides may also function as intramolecular chaperones to facilitate correct folding and the eventual targeting of these proteins to their destinations. Indeed mass-spectrometry shown in Tables 1 and 2 below, show that the LPD co-immunoprecipitates with chaperone proteins e.g., heat-shock proteins.

The LOX-PP comprises three consensus N-glycosylation sites at residues 81, 97, and 144 of the LOX protein sequence.

The present teachings contemplate, in one embodiment, a glycosylated LPD.

According to an embodiment, the polypeptide is glycosylated on at least one of N81, N97 and N144 of SEQ ID NO: 1. When referring to the glycosylation sites the indicated residues are those corresponding to SEQ ID NO: 1.

According to an embodiment, the polypeptide is glycosylated on one glycosylation site of LPD i.e., N81 of SEQ ID NO: 1.

According to an embodiment, the polypeptide is glycosylated on one glycosylation site of LPD i.e., N97 of SEQ ID NO: 1.

According to an embodiment, the polypeptide is glycosylated on one glycosylation site of LPD i.e., N144 of SEQ ID NO: 1.

According to an embodiment, the polypeptide is glycosylated on at least two of N81, N97 and N144 of SEQ ID NO: 1.

According to an embodiment, the polypeptide is glycosylated on two glycosylation sites of LPD, e.g., N81+N97 of SEQ ID NO: 1, e.g., N81+N144 of SEQ ID NO: 1 e.g., N97+N144 of SEQ ID NO: 1.

According to an embodiment, the polypeptide is glycosylated on N81, N97 and N144 of SEQ ID NO: 1.

According to an alternative embodiment, the polypeptide is not glycosylated on N81, N97 and N144 of SEQ ID NO: 1.

Alteration in glycosylation can be achieved by methods which are well known in the art e.g., any of selection of an appropriate expression system (e.g., prokaryotic vs. eukaryotic), separation of glycosylated from non-glycosylated forms, site-directed mutagenesis at the glycosylation site and/or using enzymatic modification.

The present inventors have shown that the glycosylated and non-glycosylated forms are endowed with different properties, see Example 5. For example, the non-glycosylated form has higher binding ratio against HSP70 in as determined by an immunoprecipitation experiment followed by Mass Spectrometry.

The polypeptide of some embodiments of the present invention is endowed with a number of biological activities.

According to a specific embodiment, the protein inhibits LOX in vitro and in vivo. Fc-LPD affinity and stability measurements were performed against LOX, determining the binding affinity (EC₅₀=43 nM) and dissociation constant (Kd=32 nM). In vivo studies in mdx mice showed improvement in functional tests, such as rotarod running and hanging test, as well as inhibition of fibrosis accumulation and promotion of normal collagen organization. The present results, demonstrate that inhibition of LOX by Fc-LPD is highly specific and efficiently minimizes the fibrosis and side effects of DMD.

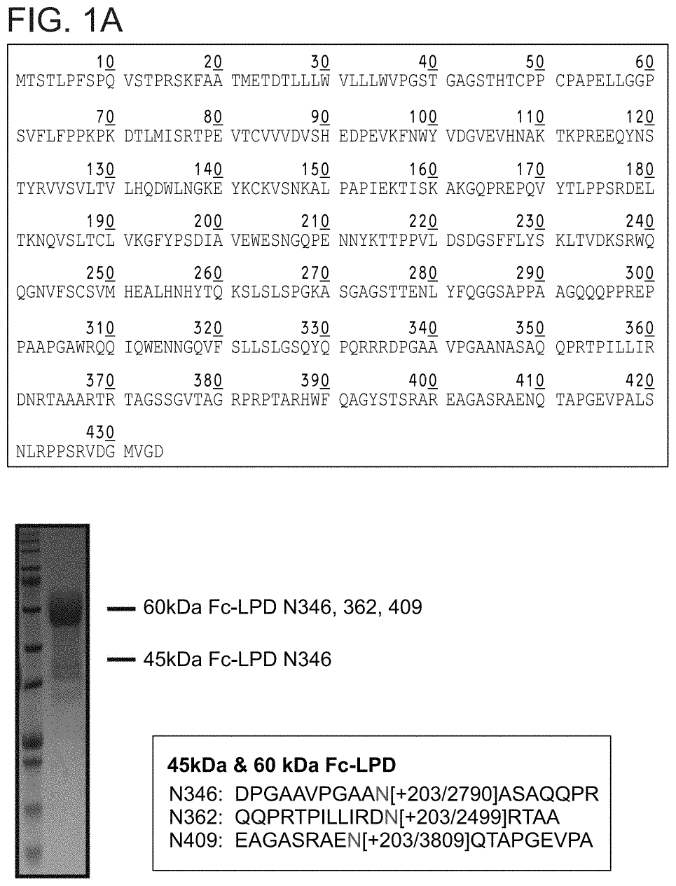

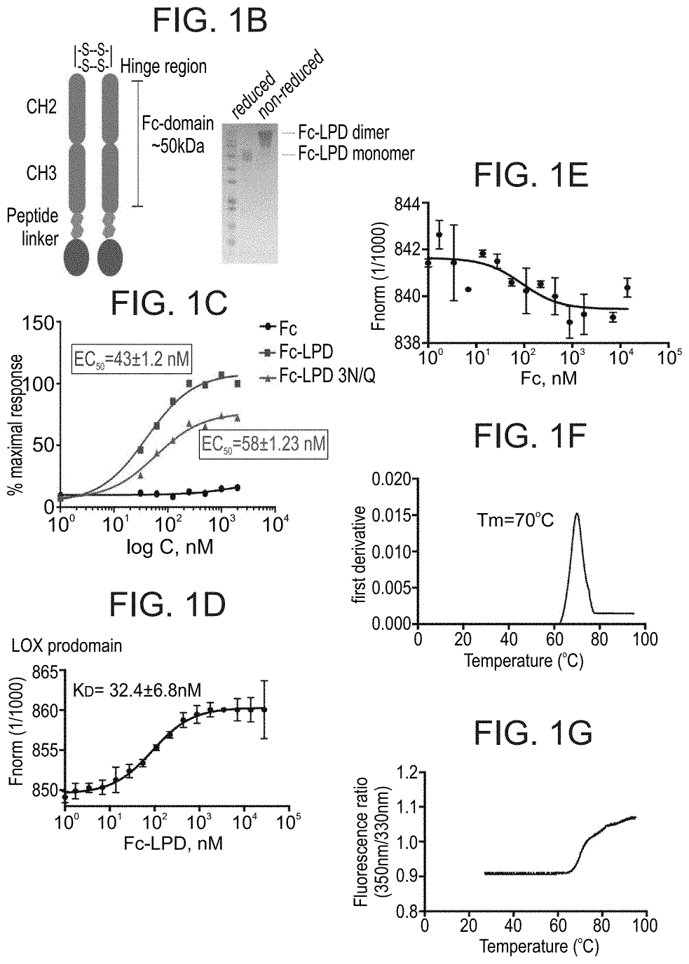

According to a specific embodiment, the polypeptide is characterized by an EC₅₀ of about 10-2000 nM, about 10-500 nM, e.g., about 43 nM, as determined by an ELISA assay (e.g., according to the assay conditions of FIG. 1C).

According to a specific embodiment, the polypeptide is characterized by a K_{D} of about 10-100 nM, e.g., about 32 nM, as determined by a microscale thermophoresis (e.g., according to the assay conditions of FIG. 1D).

According to a particular embodiment, the polypeptide is capable of down-regulating crosslinking of collagen in an in vitro assay system as further described in the Examples section hereinbelow. Since the polypeptide of some embodiments of the invention is capable of interfering with collagen crosslinking, it alters the structure of the extracellular matrix (ECM), relaxing the collagen fibers, changing them from an ordered orientation to a more random orientation. Thus, the polypeptide of some embodiments of the present invention alters the fibrillation of collagen (and accordingly strength of the collagen) and affecting the thickness of collagen.

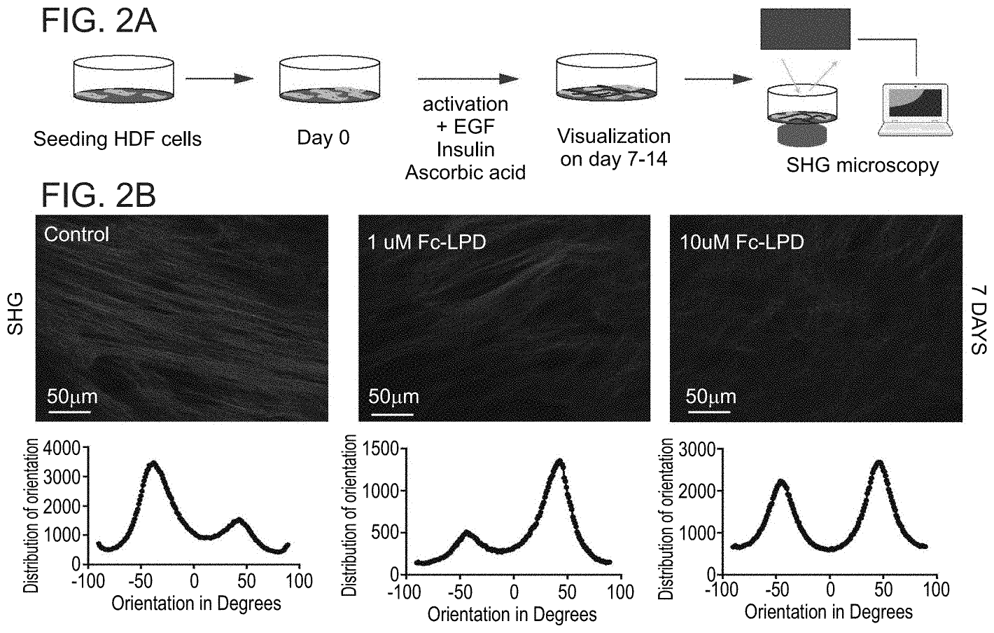

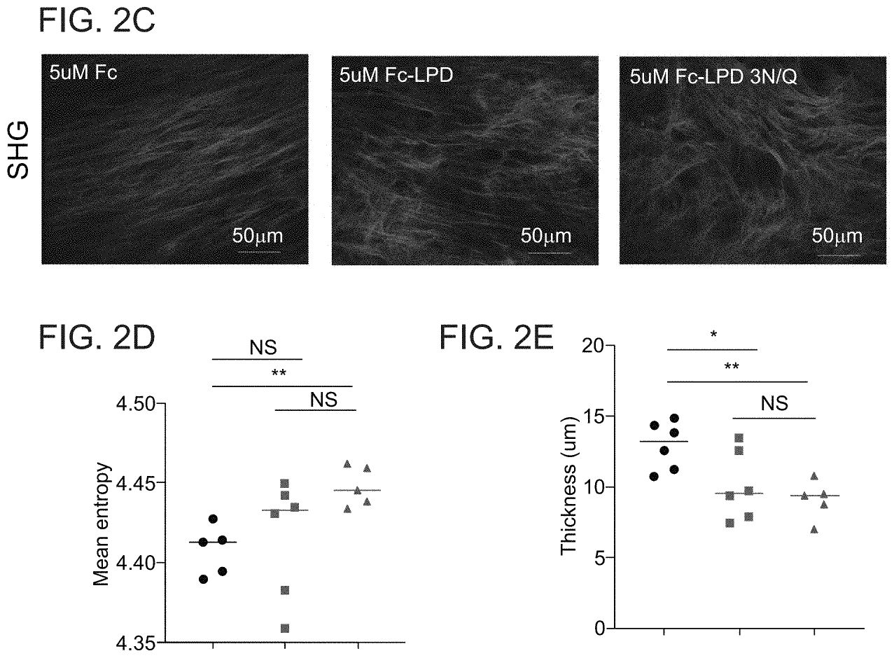

Methods of analyzing collagen architecture are known in the art and include for example second harmonic generation (SHG) microscopy (see e.g., FIGS. 2A-B-C-D), electron microscopy imaging and Raman microscopy.

Thus, according to an embodiment of the invention the polypeptide is capable of reducing at least one of fibrillar collagen (e.g., Type I) and cross-linked collagen, as determined by SHG microscopy.

By “reducing”, it is intended to mean that the amount of fibrillary/cross-linked collagen is 5%, 10%, 15%, 20%, 25%, 30%, 35%, 40%, 45%, 50%, 55%, 60%, 65%, 70%, 75%, 80%, 85%, 90%, 95%, 96%, 97% or 98% less relative to same in the absence of the polypeptide.

According to an embodiment of the invention the polypeptide is capable of altering collagen fiber orientation, as determined by SHG microscopy.

According to one embodiment, the LPD is derived (taken from or sourced) from a human LOX, although other mammalian sequences of LOX are also contemplated.

The mRNA and amino acid sequences for Homo sapiens LOX can be found under GenBank accession number P28300.2 (Protein accession number), AF039291.1 (mRNA accession number), NC_000005 (genomic accession).

As mentioned, the polypeptide is devoid of LOX catalytic activity.

As used herein “LOX catalytic activity” refers to the lysyl oxidase activity of the enzyme which is typically attributed to the domain between amino acid coordinates 169-417 of SEQ ID NO: 3 (e.g., SEQ ID NO: 9). According to a specific embodiment the catalytic activity is set forth in SEQ ID NO: 9 (amino acid sequence) and 10 (nucleic acid sequence).

Catalytic activity of LOX can be determined in vitro by SHG microscopy (described in details in the Examples section which follows).

Functional equivalents of LPD are also contemplated, having about the same or even higher activity/stability than that of SEQ ID NO: 1 or 5.

According to a particular embodiment, the LPD sequence is at least 80%, at least 85%, at least 87%, at least 89%, at least 91%, at least 93%, at least 95% or more say 100% identical to the LPD sequence described in SEQ ID NO: 1 or 5 as determined using BlastP software of the National Center of Biotechnology Information (NCBI) using default parameters) and is capable of any of the above biological functions. In measuring homology between a peptide and a protein of greater size, homology is measured only in the corresponding region; that is, the protein is regarded as only having the same general length as the peptide, allowing for gaps and insertions.

The homolog may also refer to a deletion, insertion, or substitution variant, including an amino acid substitution, thereof and biologically active polypeptide fragments thereof.

Additional modifications and changes can be made in the structure of the LPD portion of the polypeptide of the presently disclosed subject matter and still obtain a molecule being capable of inhibiting LOX. For example, certain amino acids can be substituted for other amino acids in a sequence without appreciable loss of peptide activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those which are within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e. with a biological property of the polypeptide. As detailed in U.S. Pat. No. 4,554,101, the following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); proline (−0.5.+−0.1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those which are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine. The presently disclosed subject matter thus contemplates functional or biological equivalents of the polypeptide or LPD portion thereof as set forth above.

Biological or functional equivalents of a polypeptide can be prepared using site-specific mutagenesis according to procedures well known in the art. Accordingly, amino acid residues can be added to or deleted from the LPD of the presently disclosed subject matter through the use of standard molecular biological techniques without altering the functionality of the peptide.

According to one embodiment, the amino acid sequence of the LPD is modified so as to increase its stability, bioavailability and/or pharmacological efficacy.

Thus, according to an aspect of the invention there is provided a synthetic polypeptide comprising a propeptide of human lysyl oxidase (LOX), said polypeptide being devoid of LOX catalytic activity, said synthetic polypeptide comprising a modification which imparts said polypeptide with enhanced stability under physiological conditions as compared to a native form of said polypeptide not comprising said modification.

Methods of determining stability are well known in the art.

As used herein “stability” refers to at least thermal stability. The method is based on measuring ultra-high-resolution protein stability using intrinsic tryptophan or tyrosine fluorescence.

As used herein “enhanced” or “increased” refers to an increase by at least 10%, 20%, 30%, 50%, 60%, 70%, 80%, 90% or more, say 100%, with respect to that of the native LPD. According to a specific embodiment, the polypeptide is characterized by a transition midpoint of 20-95° C. e.g., Tm about 70° C., as determined by differential scanning fluorimetry (DSF).

Thus, in order to improve the stability/activity of the LPD, the polypeptide is modified. According to a specific embodiment, the modification comprises a proteinaceous modification.

The proteinaceous modification can be attached to the polypeptide by ways of chemical attachment (fusion polypeptide such as by the use of linkers and/or active groups) or by recombinant DNA technology, whereby the synthetic polypeptide is a chimeric polypeptide.

Thus, the polypeptide has a first moiety, which is the LPD, and a second moiety, which is a heterologous peptide or protein, i.e., the proteinaceous modification. The fusion/chimera (or collectively “fusion”) can be with N to C or C to N orientation of the LPD relative to the proteinaceous moiety (also referred to herein as “heterologous polypeptide”). Fusion proteins may include myc, HA-, or His6-tags. Fusion proteins further include the LPD fused to the Fc domain of a human IgG (as referred to herein in one embodiment Fc-LPD). In particular aspects, the immunoglobulin fusion includes the hinge, CH2 and CH3, or the hinge, CH1, CH2 and CH3 regions of an IgG1 molecule. According to a specific embodiment, the Fc is as set forth in SEQ ID NO: 7. For the production of immunoglobulin fusions see also U.S. Pat. No. 5,428,130. The Fc moiety can be derived from mouse IgG1 or human IgG2_{M}4. Human IgG2_{M}4 (See U.S. Published Application No. 20070148167 and U.S. Published Application No. 20060228349) is an antibody from IgG2 with mutations with which the antibody maintains normal pharmacokinetic profile but does not possess any known effector function.

Exemplary amino acid sequences of an LPD fused to an Fc domain is set forth in SEQ ID NO: 5 (amino acid) and SEQ ID NO: 6 (nucleic acid).

Fusion proteins further include the LPD fused to human serum albumin, transferrin, or an antibody.

In further still aspects, the LPD is conjugated to a carrier protein such as human serum albumin, transferrin, or an antibody molecule.

The term “polypeptide” as used herein refers to a polymer of natural or synthetic amino acids, encompassing native peptides (either truncation products, synthetically synthesized polypeptides or recombinant polypeptides) and peptidomimetics (typically, synthetically synthesized peptides), as well as peptoids and semipeptoids which are polypeptide analogs, which may have, for example, modifications rendering the polypeptides even more stable while in a body or more capable of penetrating into cells.

Such modifications include, but are not limited to N terminus modification, C terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Polypeptide bonds (—CO—NH—) within the polypeptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), polypeptide derivatives (—N(R)—CH2-CO—), wherein R is the “normal” side chain, naturally presented on the carbon atom. These modifications can occur at any of the bonds along the polypeptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the polypeptides of the present invention may also include one or more modified amino acids or one or more non-amino acid monomers (e.g. fatty acids, complex carbohydrates etc.).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers).

Tables A and B below list naturally occurring amino acids (Table A) and non-conventional or modified amino acids (Table B) which can be used with the present invention.

TABLE A

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE B

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| Carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| Carboxylate | | L-N-methylglutamic acid | Nmglu |
| Cyclohexylalanine | Chexa | L-N-methylhistidine | Nmhis |
| Cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnory aline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α ethylcyclohexylalanine | Mchexa |
| D-αmethylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methylanapthylalanine | Nmanap |
| D-N-methylyaline | Dnmv al | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | Penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α thylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methylanapthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | Penicillamine | Pen |
| L-homophenylalanine | Hphe | Lα-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α ethylhistidine | Mhis | L-α- Mhphemethylhomophenylalanine | |
| L-α thylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-αethylvaline | Mtrp | L-α-methyltyrosine | Mtyr |
| L-α-methylleucine | Mval | L-N-methylhomophenylalanine | Nmhphe |
| | Nnbhm | | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2- | Nmbc | | |
| diphenylethylamino)cyclopropane | | | |

Recombinant techniques are typically used to generate the polypeptides (or only the LPD portion thereof) of the present invention. Such recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

To produce a polypeptide of the present invention using recombinant technology, a polynucleotide encoding the polypeptide of the present invention e.g., SEQ ID NO: 2 or 6 is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

The phrase “an isolated polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

As mentioned hereinabove, polynucleotide sequences of the present invention are inserted into expression vectors (i.e., a nucleic acid construct) to enable expression of the recombinant polypeptide. The expression vector of the present invention may include additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals). It will be appreciated that the expression vector may also comprise polynucleotide sequences encoding other polypeptides that are transcriptionally linked to the nuclear targeting peptides of the present invention. Such polypeptides are further described herein below.

Promoters used in the expression vectors may be constitutive or inducible. Tissue specific promoters are also contemplated.

A variety of prokaryotic or eukaryotic cells (e.g., HEK293-6E) can be used as host-expression systems to express the peptides of the present invention. These include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

According to this embodiment of this aspect of the present invention, the nucleic acid sequence encoding the polypeptide of the present invention may be altered, to further improve expression levels in the expression system. Thus, the polynucleotide sequence encoding the polypeptide may be modified in accordance with the preferred codon usage for bacterial or a certain mammalian expression.

The phrase “codon optimization” refers to the selection of appropriate DNA nucleotides for use within a structural gene or fragment thereof that approaches codon usage within the relevant system.

Examples of polynucleotide sequences that may be used to express the polypeptides of the present invention are provided in SEQ ID NOs: 2 and 6.

It will be appreciated that the polynucleotides of the present invention may also be expressed directly in the subject (i.e. in vivo gene therapy) or may be expressed ex vivo in a cell system (autologous or non-autologous) and then administered to the subject.

Other than containing the necessary elements for the transcription and translation of the inserted coding sequence (encoding the polypeptide), the expression construct of the present invention can also include sequences engineered to optimize stability, production, purification, yield or activity of the expressed peptide.

Various methods can be used to introduce the expression vector of the present invention into the host cell system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Transformed cells are cultured under effective conditions, which allow for the expression of high amounts of recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Depending on the vector and host system used for production, resultant polypeptides of the present invention may either remain within the recombinant cell, secreted into the fermentation medium, secreted into a space between two cellular membranes, such as the periplasmic space in E. coli; or retained on the outer surface of a cell or viral membrane.

Following a predetermined time in culture, recovery of the recombinant polypeptide is affected.

The phrase “recovering the recombinant polypeptide” used herein refers to collecting the whole fermentation medium containing the polypeptide and need not imply additional steps of separation or purification.

Recovering is also covered by the term “isolating” or “purifying”, which can also be from the host cells.

Thus, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

To facilitate recovery, the expressed coding sequence can be engineered to encode the polypeptide of the present invention and fused cleavable moiety. Such a fusion protein can be designed so that the polypeptide can be readily isolated by affinity chromatography; e.g., by immobilization on a column specific for the cleavable moiety. Where a cleavage site is engineered between the polypeptide and the cleavable moiety, the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

Exemplary purification tags for purposes of the invention include but are not limited to polyhistidine, V5, myc, protein A, gluthatione-S-fransferase, maltose binding protein (MBP) and cellulose-binding domain (CBD) [Sassenfeld, 1990, TIBTECH, 8, 88-9].

The polypeptides of the present invention are preferably retrieved in “substantially pure” form.

As used herein, the phrase “substantially pure” refers to a purity that allows for the effective use of the protein in the applications described herein.

The LPD or polypeptide (including e.g., the Fc portion) of some embodiments of the invention may be chemically modified with a chemical modification following expression for increasing bioavailability.

Thus, for example, the present invention contemplates modifications wherein the polypeptide is linked to a polymer. The polymer selected is usually modified to have a single reactive group, such as an active ester for acylation or an aldehyde for alkylation, so that the degree of modification may be controlled. Included within the scope of polymers is a mixture of polymers. Preferably, for therapeutic use of the end-product preparation, the polymer will be pharmaceutically acceptable.

The polymer or mixture thereof may be selected from the group consisting of, for example, polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In further still embodiments, the polypeptide is modified by PEGylation, HESylation CTP (C terminal peptide), crosslinking to albumin, encapsulation, modification with polysaccharide and polysaccharide alteration. The modification can be to any amino acid residue in the polypeptide.

According to one embodiment the modification is to the N or C-terminal amino acid of the LPD. This may be affected either directly or by way coupling to the thiol group of a cysteine residue added to the N or C-terminus or a linker added to the N or C-terminus such as Ttds. In further embodiments, the N or C-terminus of the polypeptide comprises a cysteine residue to which a protecting group is coupled to the N-terminal amino group of the cysteine residue and the cysteine thiolate group is derivatized with a functional group such as N-ethylmaleimide, PEG group, HESylated CTP.

It is well known that the properties of certain proteins can be modulated by attachment of polyethylene glycol (PEG) polymers, which increases the hydrodynamic volume of the protein and thereby slows its clearance by kidney filtration. (See, for example, Clark et al., J. Biol. Chem. 271: 21969-21977 (1996). Therefore, it is envisioned that the core peptide residues can be PEGylated to provide enhanced therapeutic benefits such as, for example, increased efficacy by extending half-life in vivo. Thus, PEGylating the polypeptide will improve the pharmacokinetics and pharmacodynamics of the propeptide domain of the polypeptide.

PEGylation methods are well known in the literature and described in the following references, each of which is incorporated herein by reference: Lu et al., Int. J. Pept. Protein Res. 43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., Int. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C.sub.1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG.sub.2-NHS-40k (Nektar); mPEG2-MAL-40k (Nektar), SUNBRIGHT GL2-400MA ((PEG).sub.240 kDa) (NOF Corporation, Tokyo), SUNBRIGHT ME-200MA (PEG20 kDa) (NOF Corporation, Tokyo). The PEG groups are generally attached to the LPD polypeptide via acylation, amidation, thioetherification or reductive alkylation through a reactive group on the PEG moiety (for example, an aldehyde, amino, carboxyl or thiol group) to a reactive group on the polypeptide (for example, an aldehyde, amino, carboxyl or thiol group).

The PEG molecule(s) may be covalently attached to any Lys or Cys residue at any position in the polypeptide. Other amino acids that can be used are Tyr and His. Optional are also amino acids with a Carboxylic side chain. The polypeptide described herein can be PEGylated directly to any amino acid at the N-terminus by way of the N-terminal amino group. A “linker arm” may be added to the polypeptide to facilitate PEGylation. PEGylation at the thiol side-chain of cysteine has been widely reported (See, e.g., Caliceti & Veronese, Adv. Drug Deliv. Rev. 55: 1261-77 (2003)). If there is no cysteine residue in the polypeptide, a cysteine residue can be introduced through substitution or by adding a cysteine to the N-terminal amino acid. Other options include reagents that add thiols to polypeptides, such as Traut's reagents and SATA.

In particular aspects, the PEG molecule is branched while in other aspects, the PEG molecule may be linear. In particular aspects, the PEG molecule is between 1 kDa and 150 kDa in molecular weight. More particularly, the PEG molecule is between 1 kDa and 100 kDa in molecular weight. In further aspects, the PEG molecule is selected from 5, 10, 20, 30, 40, 50 and 60 kDa.

A useful strategy for the PEGylation of a polypeptide consists of combining, through forming a conjugate linkage in solution, a peptide, and a PEG moiety, each bearing a special functionality that is mutually reactive toward the other. The polypeptide can be easily prepared by recombinant means as described above.

According to one embodiment, the PEG is “preactivated” prior to attachment to the polypeptide. For example, carboxyl terminated PEGs may be transformed to NHS esters for activation making them more reactive towards lysines and N-terminals.

According to another embodiment, the polypeptide is “preactivated” with an appropriate functional group at a specific site. Conjugation of the polypeptide with PEG may take place in aqueous phase or organic co-solvents and can be easily monitored by SDS-PAGE, isoelectric focusing (IEF), SEC and mass spectrometry. The PEGylated polypeptide is then purified. Small PEGs may be removed by ultra-filtration. Larger PEGs are typically purified using anion chromatography, cation chromatography or affinity chromatography. Characterization of the PEGylated polypeptide may be carried out by analytical HPLC, amino acid analysis, IEF, analysis of enzymatic activity, electrophoresis, analysis of PEG:protein ratio, laser desorption mass spectrometry and electrospray mass spectrometry.

Removal of excess free PEG may be performed by packing a column (Tricorn Empty High-Performance Columns, GE Healthcare) with POROS 50 HQ support (Applied Biosystems), following which the column is equilibrated with equilibration buffer (25 mM Tris-HCl buffer, pH 8.2). The PEGylated polypeptide is loaded onto the equilibrated column and thereafter the column is washed with 5CV of equilibration buffer. Under these conditions, the polypeptide binds to the column. PEGylated polypeptide is eluted in the next step by the elution buffer and stored at 2-8° C. for short term, or frozen at −20° C. for long term storage.

The resultant polypeptide may be anywhere between 40-100 KDa, dependent on whether a modification has been added (as described above) and on the glycosylation status (as described above).

For instance, in the case of an Fc fusion:

According to a specific embodiment, the polypeptide is 40-70 KDa.

According to a specific embodiment, the polypeptide is 45-65 KDa.

According to a specific embodiment, the polypeptide is 50-70 KDa.

According to a specific embodiment, the polypeptide is 45 KDa.

According to a specific embodiment, the polypeptide is 65 KDa.

According to a specific embodiment, the polypeptide domain of LOX devoid of the LOX catalytic activity is below 400 amino acids e.g., 100-200, 100-150, 100-300 amino acids long.

The synthetic polypeptide can be used in any method of inhibiting catalytic (enzymatic activity) of LOX, by mere contacting the LOX with the synthetic polypeptide comprising the modification. Such methods can be performed in-vitro, in-vivo or ex-vivo.

Polypeptides of some embodiments of the invention can be used in treating fibrosis or fibrotic conditions as mentioned hereinabove.

According to a specific embodiment, the polypeptides of some embodiments of the present invention are not used (or administered) as part of a regimen, which comprises treatment with D-penicillamine.

The polypeptide or polynucleotide encoding same of the present invention can be provided to the treated subject (i.e. mammal) per se (e.g., purified or directly as part of an expression system) or can be provided in a pharmaceutical composition comprising the polypeptide of the present invention. As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polypeptide (or polynucleotide encoding same) of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable peripheral routes of administration may, for example, include oral, rectal, transmucosal, especially transnasal, intestinal or parenteral delivery, including intramuscular, subcutaneous and intravenous, intraperitoneal, intranasal, or intraocular injections.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use. The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

The Examples section which follows describes in details animal models for DMD as well as standard tests to determine efficacy.

Animal models for other fibrotic conditions are well known in the art, see e.g., liver fibrosis models are described in Delire et al. J Clin Transl Hepatol. 2015 March; 3(1): 53-66; Moore et al describe animal models for fibrotic lung diseases; and Rai et al. Mol Cell Biochem. 2017 January; 424(1-2): 123-145. doi: 10.1007/s11010-016-2849-0. Describe animal models for cardiac fibrosis.

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is affected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an U.S. Food and Drug Administration (FDA) approved kit, which may contain one or more-unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration.

Such notice, for example, may be of labeling approved by the U.S. Food and Drug Administration (FDA) for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Material and Methods

Fc-LPD Expression and Purification

The prodomain domain of human LOX (22-168) SEQ ID NO: 1: was cloned into pYD5 mammalian expression vector and the plasmid was transformed into competent HEK 293-6E cells in an exponential growth phase with polyethylenimine (PEI) transfection reagent in a DNA-PEI ratio of 1:2. After 120 hours incubation the cells were harvested and the supernatant was purified on a HiTrap Protein A column, equilibrated with 100 mmol/L phosphate buffer (pH 8) and the elution was performed by 100 mmol/L citrate buffer (pH 3-4.5).

LOX Catalytic Domain Expression and Purification

The catalytic domain of human LOX (169-417) of SEQ ID NO: 3 i.e., SEQ ID NO: 10 was cloned into the pET28 expression vector and transformed into competent Escherichia coli BL21 SHuffle strain. A single colony was resuspended in 10 ml liquid medium supplemented with antibiotic. The 10 ml culture was used to inoculate 1 L of liquid medium. The culture was incubated at 30° C. until O.D₆₀₀ reached 0.4-0.8 after which 0.4 mM IPTG inducer was added. The culture was incubated overnight 16° C., 250 rpm. The medium was centrifuged for 15 min in 4000 rpm. The pelleted fractions were frozen for 30 min following incubation on ice with lysis buffer containing 50 mM Tris-HCl (pH 8), 200 mM NaCl, 40 mM Imidazole, lysozyme, protein inhibitor and DNase. The solution was sonicated and centrifuged at 24,000 rpm for 40 min. The suspension was purified with 0.22 μM filter and loaded on to a HisTrap column (GE Healthcare), which was pre-equilibrated with the following buffer: 50 mM Tris-HCl pH 8, 200 mM NaCl, 40 mM Imidazole. The protein was eluted with 50 mM Tris-HCl pH 8, 200 mM NaCl, 400 mM Imidazole. The enzyme was purified by size-exclusion chromatography using a HiLoad 16/60 Superdex 75 (Amersham Biosciences) and eluted with 50 mM Tris-HCl pH 8, 200 mM NaCl.

Collagen Assembly Assay

HDF cells were plated in 35 mm dishes in DMEM 10% FCS medium. The day after in cell culture medium the following factors were added for induction of extracellular matrix expression, 5 ng/mL EGF, 5 ug/mL insulin, and 150 ug/mL L-ascorbic, in the first experiment in the presence (1 uM and 10 uM) or the absence of Fc-LPD and for the second experiment in the presence of Fc, Fc-LPD and Fc-LPD N346, 362, 409Q (5 uM). The ECM formation was monitored from 1 to 2-weeks with two-photon microscopy and second harmonic generation (FIGS. 2B-C) (see below).

ELISA Binding Assay

A ninety-six-well plate (Nunc) was coated with LOX-catalytic domain, HSP70 or BSA at 10 μg/mL. After blocking with 2% BSA in PBS, the plate was incubated with Fc and Fc-LPD for 1 hour at 37° C. Bound antibodies were detected by peroxidase-conjugated antibody goat anti-human (Jackson ImmunoResearch). EC₅₀ was calculated with GraphPad Prism from Find ECanything curve fitting analysis.

Microscale Thermophoresis (MST) LOX (200 nM) (SEQ ID NO: 9) was Labeled with Monolith His-Tag Labeling Kit Using NT-647 dye (RED-tris-NTA) and incubated for 30 minutes. Concentration of Fc and Fc-LPD was increased and samples were measured using a Monolith NT.115 (Nanotemper, Germany) with 50% Laser-power, laser on time of 35 seconds and 40% LED-power.

Differential Scanning Fluorimetry (DSF)

The NanoDSF Prometheus NT.48 measuring ultra-high-resolution of LOX protein stability using intrinsic tryptophan or tyrosine fluorescence by continuous heating the samples with an adjustable heating rate of 1° C./min in a range of 20-95° C. and simultaneously reads both the fluorescence and back reflection signal.

Immunoprecipitation

Fc-LPD was covalently coated on protein G beads (GE) according to manufacturer's instructions (GE I.P beads). The coated beads were incubated with a concentrated supernatant from 10 cm² dish culture of HDF cells overnight on 4° C. Pellet beads were obtained by centrifugation (˜500×g for 5 min 4° C.) and the beads were washed with PBS of volume at least 5 times the initial beads volume. Then the beads pellet was placed in a collection column, which was inserted in an eppendorf tube containing 10 ul of 1M Tris-HCl pH 8. 90 μl of elution buffer (Thermo-Scientific) was added to the beads and after 5 min of incubation elution was performed by centrifuge. The last step was repeated four times.

Mass-Spectrometry for Characterization of Fc-LPD

The gel bands were subjected to in-gel tryptic digestion, followed by a desalting step. The resulting peptides were analyzed using nanoflow liquid chromatography (nanoAcquity) coupled to high resolution, high mass accuracy mass spectrometry (Fusion Lumos), using EThcD fragmentation to generate fragmentation of both glycans and peptides. Each sample was analyzed separately in a random order in discovery mode. The data was processed in Byonic v2.15.89. This is a unique software that can process such data and identify glycans. The search was done against the uniprot human database and the following modifications allowed: Carbamidomethylation of C as a fixed modification, carbamidomethylation of N-term, H or K, deamidation of N or Q, Glu or Gln to pyro-Glu, ammonia loss, N-term acetylation, Formylation of STK, dehydration of ST and N-glycosylation as variable ones. The data was filtered to allow a maximum of 1% false discovery rate (FDR).

Mass-Spectrometry for Identification Gel Bands from I.P of Fc-LPD

The gel bands were subjected to in-gel tryptic digestion, followed by a desalting step. The resulting peptides were analyzed using nanoflow liquid chromatography (nanoAcquity) coupled to high resolution, high mass accuracy mass spectrometry (Q Exactive Plus). Each sample was analyzed on the instrument separately in a random order in discovery mode. The data was processed using Proteome Discoverer version 2.2.0.388, searched against the Uniprot human protein database, to which a list of common lab contaminants was added. The search was done with two search algorithms—SequestHT and Mascot.

Two-Photon Microscopy and Second Harmonic Generation

Images of the samples were taken using 2 PM: Zeiss LSM 510 META NLO microscope equipped with a broadband Mai Tai-HP-femtosecond single box tunable Ti-sapphire oscillator, with automated broadband wavelength tuning 700 to 1,020 nm from Spectra-Physics using an 855-nm wavelength (detection at 400 nm) with 20× & 25× objectives. The thickness of the ECM was calculated based on z-stack in um.

Image Analysis

Imaging analysis was done by Fiji package [28]. Fourier component analysis for directionality was performed on data using the Fiji plug-in “Directionality” created by Jean-Yves Tinevez (pacificdotmpi-cbgdotde/wiki/indexdotphp/Directionality) and following Fiji's instructions. For entropy calculation was used the Fiji macro CDS.ijm to create orientation analysis files and afterwards mean entropy was calculated by matlab script developed by O. Golani & G. Molodij and modified by E. Shimshoni.

Animal Experiments

For early stage experiment male Mdx mice (8 per group) approximately 8 weeks old were used. One group was treated with 5 mg/kg Fc, the second group with 5 mg/kg Fc-LPD and third with 5 mg/kg Fc-LPD N346, 362, 409Q once a week with intraperitoneal injection. Every two weeks the mice were tested in rotarod assay. Mice were sacrificed after 3 months and heart, diaphragm, tibialis anterior, quadricep and gastrocnemius muscle was immediately fixed or frozen in OTC.

For late stage experiment male Mdx mice (4 per group) approximately 23-25 weeks old were used. One group was treated with 5 mg/kg Fc and the other group with 5 mg/kg Fc-LPD once a week with intraperitoneal injection. Every two weeks the mice were tested in rotarod assay and every other week they were tested with hanging test with four limbs. Mice were sacrificed after 3 months and heart, diaphragm, tibialis anterior and gastrocnemius muscle was immediately fixed or frozen in OTC.

Rotarod Running

For rotarod running assay the muscle strength, coordination, balance, and condition were determined. Mice were placed on the tube of the rotarod when it rotated at a slow steady speed of 5 rpm. After mice were positioned, the speed for the first 15 sec was accelerated from 5 to 45 rpm and then it was maintained at that speed. The running time was continuously recorded by the software and stopped automatically when a mouse fell off the tube. If the mice turned facing to the opposite direction on the tube while running, reposition was performed without stopping the rotation of the tube.

Hanging Test

With hanging test, balance, coordination and muscle condition can be assessed. This test was based on the knowledge that mice are eager to remain hanging on a wire or grid till exhaustion. Specifically, the lid of a big cage for a rat was used. The mouse via tail handling was brought near the grid. The mouse was let to grasp the grid with the four paws. The grid was inverted so that the mouse was hanging and directly the timer was started. After the mouse fell off the wire the timer was stopped and record of the hanging time was marked.

Histochemistry

Two different tissue preparation protocols (paraffin embedded and microtomed frozen sections) were applied, as previously described (Rolls et al., 2008). The slides were exposed to Hoechst stain (1:4,000; Invitrogen Probes) for 1 min. Sirius Red staining was performed to label Collagen type I and III, Reticulin staining for Collagen type III and Masson's trichrome stain for distinguishing cells from surrounding connective tissue. For microscopic analysis, a Nikon light microscope (Eclipse E800) equipped with a Nikon digital camera (DS-Ri1) was used.

Yeast Surface Display Expression

The constructs were amplified and cloned into pETCON (LPD) plasmids, in frame with the AGA2 gene, an N-terminal HA tag and a C-terminal Myc tag. The constructs were then transformed into EBY100 (Saccharomyces cerevisiae) strain and plated on SD-Trp plates for selection. After 48 h in 30° C. incubation, single colonies were subject to colony PCR. Fragments that met the expected band size were PCR purified and sequenced.

Constructs were cultured in SD-CAA¹⁴³ medium at 30° C., up to 0.8 OD₆₀₀, at that point the cells were pelleted and induced in a fresh SG-CAA¹⁴³ medium for 38 h in 15° C. Displayed protein expression was measured with two complementary analyses: Flow cytometry and activity assay (for wt constructs).

3×10⁷ of fully induced cells were pelleted and washed for two consecutive washes. Each wash consists of resuspension in 0.8 ml of PBS+0.5% BSA buffer (washing buffer), centrifugation for 30 sec and 13 kRPM. Washed cells were incubated with an α-c-Myc antibody (Santa Cruz 9e10, diluted 1:50) in 200 μl of wash buffer for 45 min at RT, and washed twice. The cells were further labeled with a secondary goat α mouse—IgG (Fc specific)—FITC antibody (Sigma F4143, diluted 1:50) for an additional 30 min in 200 μl at 4° C. 3.3×10⁶ EBY100 cells, after expression and α-c-Myc/FITC labeling, were washed twice, supplemented with biotinylated peptides (30-100 μM) in 200 μl of washing buffer, and incubated in RT for 2 h. Following incubation, the cells were washed twice with wash buffer and incubated on ice for an additional 30 min with 1:100 diluted streptavidin-APC (Allophycocyanin-Streptavidin, 0.5 mg/ml, Jackson ImmunoResearch) in 100 μl of wash buffer. For LPD-HSP70 interaction, cells were incubated for 30 min in RT with HSP70-labeled with Atto 633 dye (Sigma 51253). Finally, cells were washed twice, filtered through 70 μm mesh nylon strainer, and diluted in 1.6 ml of wash buffer. These were followed by flow cytometry analysis and the data were analysed by Flow Jo software.

Statistical Analyses

All analyses were performed at least triplicate. Statistical analysis was performed using GraphPad Prism 7.

Materials and Methods for the glycosylation mutants.

### Example 1

### Expression and Characterization of Fc-LPD

To dissect the extracellular LOX activity in DMD and clear fibrosis during the disease progression, a protein engineered inhibitor with the endogenous inhibitor of LOX was developed. Specifically, the auto-inhibitory LPD (SEQ ID NO: 1 encoded by nucleotide sequence set forth in SEQ ID NO: 2) was selected to inhibit the activated form of LOX (FIG. 1A). Based on the fact that the prodomains are not stable and their production faces difficulties, LPD fused to Fc-tag (SEQ ID NO: 7 encoded by nucleotide sequence set forth in SEQ ID NO: 8) was generated in order to increase the functionality. Importantly, the Fc-LPD (SEQ ID NO: 5 encoded by nucleotide sequence set forth in SEQ ID NO: 6) is expressed in dimer formation, creating a minimized antibody-like inhibitor of approximately 95 kDa molecular size (FIG. 1B). After expression in mammalian cells, HEK293-6E, the cell culture media was purified by protein A column and the pure Fc-LPD was dialyzed in PBS before starting the evaluation of functional assays.

The evaluation of the binding affinity and inhibitory activity of Fc-LPD was performed by estimating the binding affinity by measuring the EC₅₀ and the dissociation constant (K_{D}), respectively. EC₅₀ of generated Fc-LPD inhibitor was measured by ELISA (FIG. 1C) ranged from 10 to 2000 nM against the LOX catalytic domain (EC₅₀=43 nmol/L). The dissociation constant (K_{D}) of Fc-LPD (27 nmol/L) was determined by microscale thermophoresis (FIG. 1D), while no interaction was detected on Fc region of the inhibitor. Ultra-high-resolution stability of Fc-LPD was measured with differential scanning fluorimetry (DSF) and the transition midpoint was identified at 70° C. indicating that the Fc-LPD is stable in solution (FIGS. 1F, 1G).

After purification of the Fc-LPD the SDS-page analysis of the samples, either in reduced or non-reduced conditions, indicated a mixture population. The LPD is known for three different N-glycosylation sites and since the expression was performed in mammalian expression system the different size bands (45 kDa and 60 kDa) were analysed by MS. The results showed that N81-glycosylation (N346 in the present construct) was detected in both bands with multiple glycoforms in range from 203-2790 Da in 45 kDa Fc-LPD & 60 kDa band (FIG. 1A), but with higher frequency in the case of 60 kDa band. The N144-glycosylation (N409 in the construct) site was detected again in both 45 & 60 kDa Fc-LPD with glycans ranging at 203-3809 Da. The N97-glycosylation site (N362 in the construct) was detected in both 45 & 60 kDa bands only after digestion with a-lytic protease (Sigma A6362) in range from 203-2499 Da in 45 kDa Fc-LPD & 60 kDa band. Finally, one more glycosylation site in the Fc region of both bands was identified, bearing a few glycoforms of 1300-1800 Da in size. In FIG. 1A the amino acid positions have shifted N81,97,144 are now N346,362, 409 because of the presence of the Fc (SEQ ID NO: 5).

### Example 2

### Identification of Fc-LPD Capability of Interfering with Collagen Fibers Assembly

The effect of Fc-LPD on the enzymatic activity of LOX was examined in in vitro system using activated human dermal fibroblast (HDF). In detail, a screening system was developed to monitor specific events and stages of collagen cross-linking reactions by advanced microscopic tools (FIG. 2A). HDF cells were cultured with activation media containing EGF, insulin and L-ascorbic acid, and expression of ECM molecules, mostly collagen, was induced. Monitoring assembly of fibrillary collagen by two-photon SHG microscopy, with and without Fc-LPD, it was identified that the newly designed Fc-LPD targets advanced stages of collagen crosslinking and assembly. These results demonstrate that inhibition of LOX-mediated collagen cross-linking by Fc-LPD changes the orientation of normal collagen fibril alignment in vitro in a native fibroblast-derived 3D matrix scaffold (FIG. 2B).

Furthermore, HDF cells were cultured with ECM activation media in the presence of Fc, Fc-LPD and Fc-LPD N346, 362, 409Q (5 uM). After 7 days of culture the collagen fibers were monitored with two-photon SHG microscopy and disruption in fibers orientation was observed (FIG. 2C). Significant increase of entropy in the system which contained Fc-LPD N346, 362, 409Q was demonstrated (FIG. 2D). Moreover, measurement of ECM thickness (z-stack) highlighted the significant reduction of ECM production on Fc-LPD- and Fc-LPD N346, 362, 409Q-treated cells (FIG. 2E).

### Example 3

### Interactions of Different Fc-LPD Glycosylation Forms

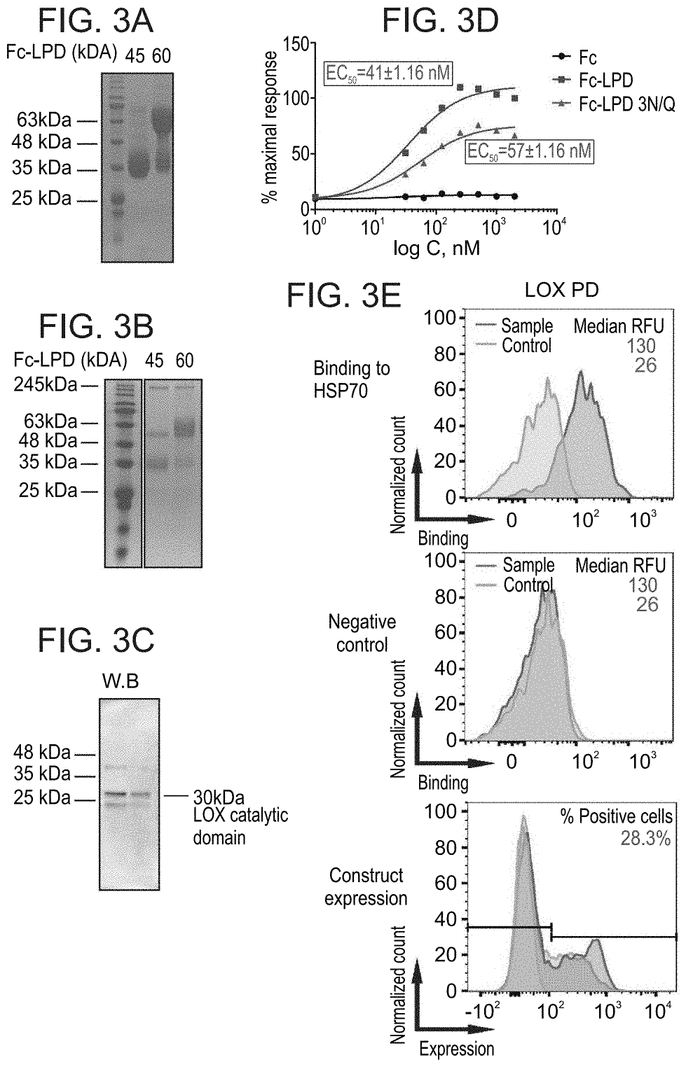

The interactions of Fc-LPD with proteins secreted by HDFs were examined by an immunoprecipitation experiment using the supernatant from HDF cells. For this purpose, HDF cells were cultured in 60 cm² dishes for 3 days in 10 ml serum-free and phenol red-free medium. The medium was collected and concentrated using 10 kDa cutoff vivaspin filters and incubated with 45 kDa Fc-LPD and 60 kDa Fc-LPD, separately, which was previously semi-separated by gel filtration and incubated with Protein G beads (FIG. 3A). The samples were analysed by SDS-page and western blot. In SDS-page analysis of the two samples, two high molecular weight bands were detected and were analysed by MS (FIG. 3B), whereas in a Western blot experiment the LOX (30 kDa) was detected, highlighting the ability of Fc-LPD to bind LOX in physiological conditions in vitro (FIG. 3C). Analysis of MS data showed the interactions of both Fc-LPD forms with ECM molecules, which are either part of core matrisome or matrisome-associated. Specifically, among the co-immunoprecipitated proteins was ECM glycoproteins (fibronectin and thrombospondin-1), ECM regulators (SerpinB12, A2-macroglobulin), secreted factors (filaggrin-2, hornerin), extracellular region or secreted proteins (HSP70, a2-HS-glycoprotein, etc.) and other proteins (Table 1 below). These interactions are direct interactions with Fc-LPD. Since HSP70 pinpointed as an important interactor, evaluation of binding affinity of Fc-LPD & Fc-LPD N346, 362, 409Q were performed by estimating the binding affinity by measuring the EC₅₀. EC₅₀ of generated Fc-LPD & Fc-LPD N346, 362, 409Q inhibitors was measured by ELISA (FIG. 3D) ranged from 10 to 2000 nM against the HSP70 (EC₅₀=41 nmol/L). Moreover, the direct binding and the high affinity of this interaction were proved with yeast surface display. Yeast cells expressed the LPD on the cell surface and labelled HSP70 was added to the solution. After 30 min of incubation in RT the interaction was measured by flow cytometry and significant population of LPD positive cells were positive for binding to HSP70 (FIG. 3E).

TABLE 1
Summary of proteins identified by mass spectrometry

| | | | | | Protein | |
|---|---|---|---|---|---|---|
| | | | | | coverage | |
| | | | | | by | |
| | | | | | amino | |
| | | | Molecular | No. of | acid | |
| | | | Mass | unique | count | |
| | UniProtKB | Name | (kDa) | peptides | (%) | Gene |
| ECM | P98095 | Fibulin-2 | 126.5 | 1 | 2 | FBLN2 |
| glycoproteins | P07996 | Thrombospondin-1 | 129.3 | 1 | 1 | THBS1 |
| | P14543 | Nidogen-1 | 136.3 | 1 | 1 | NID1 |
| | P02751 | Fibronectin | 262.5 | 6 | 4 | FN1 |
| | Q12805 | EGF-containing | 54.6 | 2 | 4 | EFEMP1 |
| | | fibulin-like | | | | |
| | | extracellular | | | | |
| | | matrix protein 1 | | | | |
| ECM | Q96P63 | Serpin B12 | 46.2 | 1 | 2 | SERPINB12 |
| regulators | P01023 | Alpha-2- | 163.2 | 3 | 2 | A2M |
| | | macroglobulin | | | | |
| Secreted | Q5D862 | Filaggrin-2 | 247.9 | 1 | 0 | FLG2 |
| factors | Q86YZ3 | Hornerin | 282.2 | 1 | 2 | HRNR |
| Extracellular | P02765 | Alpha-2-HS- | 39.3 | 2 | 5 | AHSG |
| region or | | glycoprotein | | | | |
| secreted | P01024 | Complement C3 | 187 | 1 | 1 | C3 |
| | P81605 | Dermcidin | 11.3 | 1 | 10 | DCD |
| | P0DMV9 | Heat shock 70 | 70 | 7 | 15 | HSPA1B |
| | | kDa protein 1B | | | | |
| | P12273 | Prolactin- | 16.6 | 1 | 8 | PIP |
| | | inducible protein | | | | |

### Example 4

### In Vivo Evaluation of DMD Muscle Fibrosis after Treatment with Fc-LPD & Fc-LPD N346, 362, 409Q

To test whether the LOX inhibitor could attenuate muscle fibrosis in vivo in a DMD background and thus if it could be used as a novel route for treating DMD or other fibrotic diseases, two experiments with mdx mice were carried out.

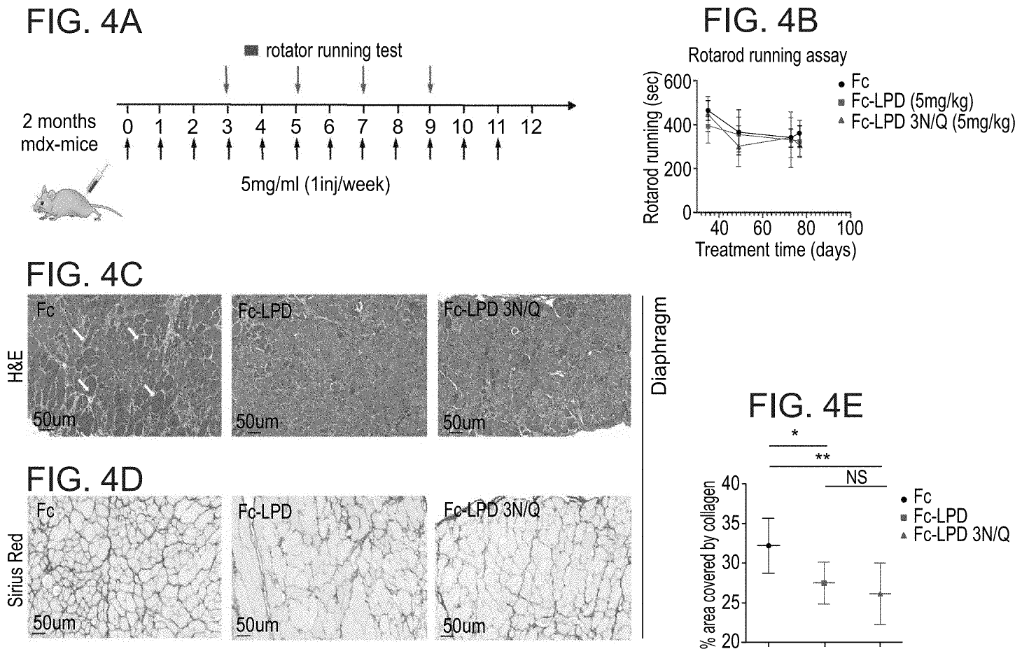

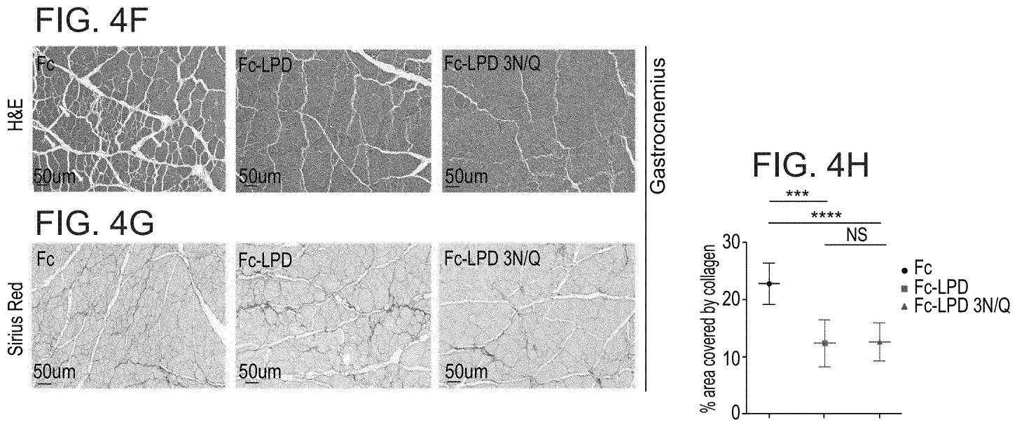

On the early stage model 2 months old male mdx mice were injected once a week with 5 mg/kg of the LOX inhibitors (Fc-LPD & Fc-LPD N346, 362, 409Q) or a control antibody (Fc) (n=8 for each group) (FIG. 4A). During the treatment functional muscle assay was performed, such as rotarod running, but not a significant improvement was observed after treatment with Fc-LPD& Fc-LPD N346, 362, 409Q (FIG. 4B). Three months after treatment had commenced (total of 11 anti-LOX injections), mice were harvested and a histological analysis of muscle fibrosis (cardiac, quadricep, gastrocnemius, tibialis anterior and diaphragm) was carried out using Sirius red staining and two-photon second harmonics generation microscopy, which monitored the native collagen fibers.

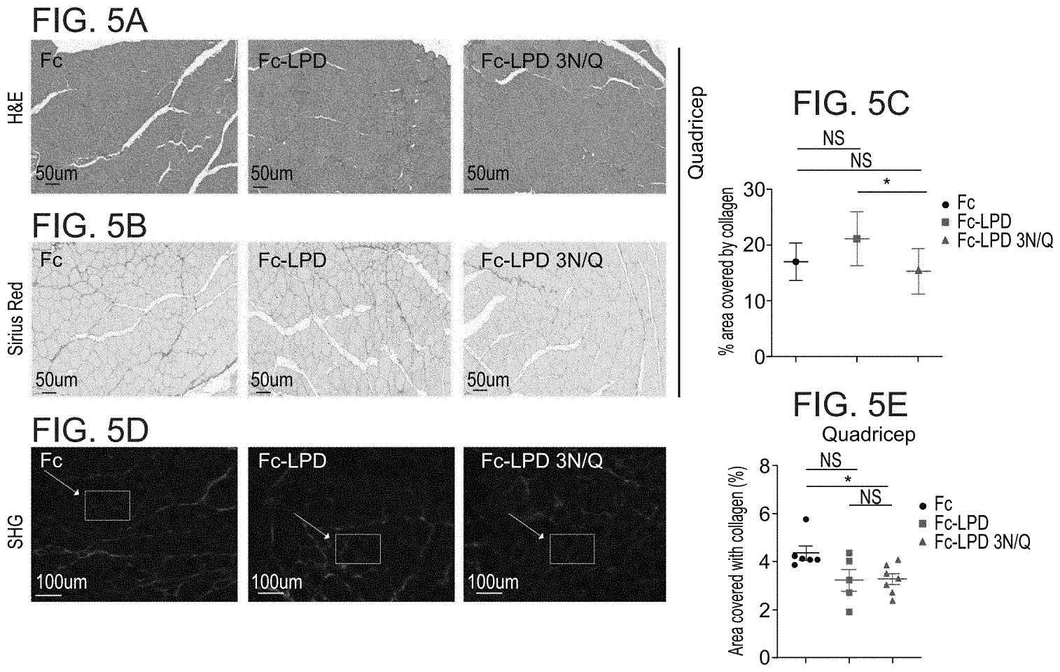

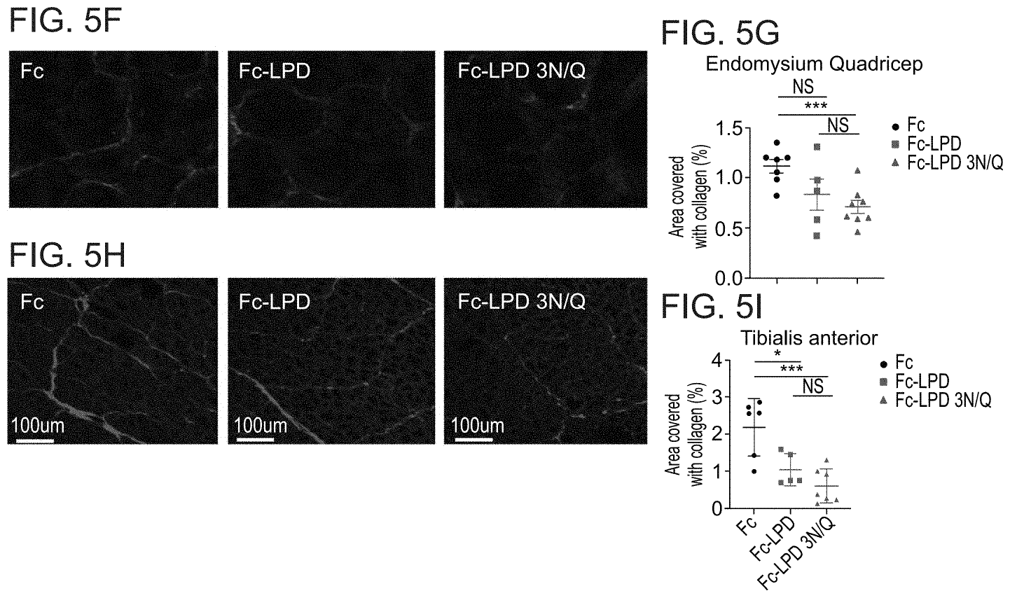

Specifically, H&E staining in diaphragm tissue showed significant improvement of central nuclei pathologic phenotype, which is observed in DMD pathological tissues, after treatment with Fc-LPD & Fc-LPD N346, 362, 409Q (FIG. 4C). Moreover, sirius red staining of diaphragm showed ˜15% & ˜20% significant reduction in fibrillary collagen deposition in the Fc-LPD & Fc-LPD N346, 362, 409Q mice, respectively (FIGS. 4D-E). Interestingly, in the case of gastrocnemius muscle significantly less centrally nucleated myofibers was observed (FIG. 4F). The collagen deposition after staining with sirius red showed ˜45% reduction in the case of Fc-LPD & Fc-LPD N346, 362, 409Q compare to Fc control (FIGS. 4G-H). By examining the quadricep tissue, no significant reduction in the amount of collagen deposition between the anti-LOX-treated mice and Fc-treated was observed (FIGS. 5A, 5B, 5C). However, further monitoring of fibrillar collagen of quadricep tissue by two-photon SHG microscopy after treatment with Fc-LPD N346, 362, 409Q showed significant reduction of collagen fibers signal (˜26%). The reduction of collagen fibers in Fc-LPD N346, 362, 409Q-treated mice was significant, when the analysis took place only on the endomysium of quadricep muscle (˜34%). The last tissue was analysed in the early stage model by two-photon SHG analysis was tibialis anterior. Collagen fibers was reduced by ˜53% for the Fc-LPD-treated mice and ˜76% for the Fc-LPD N346, 362, 409Q-treated mice compare to control Fc.

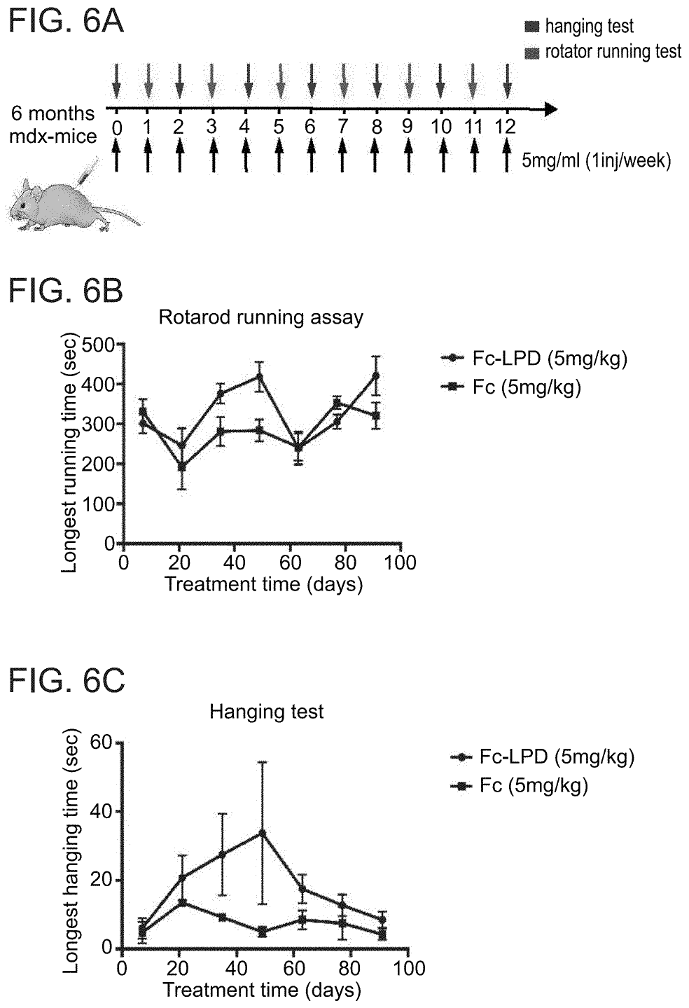

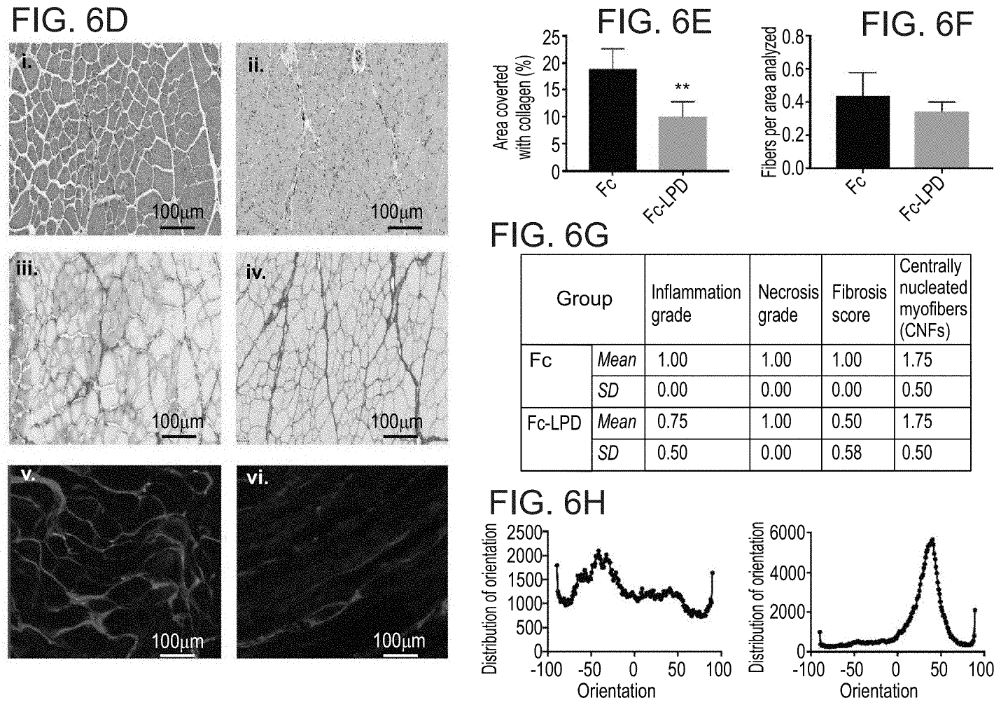

For the later stage 6 months old male mdx mice were injected once a week with 5 mg/kg of the LOX inhibitor (Fc-LPD) or a control antibody (Fc) (n=4 for each group) (FIG. 6A). During the treatment functional muscle assays were performed, such as rotarod running and hanging test, and a significant improvement was observed after Fc-LPD treatment (FIGS. 6B, 6C). Two months after treatment had commenced (total of 8 anti-LOX injections), mice were harvested and a histological analysis of muscle fibrosis (cardiac, tibialis anterior and diaphragm) was carried out using Sirius red staining and two-photon second harmonics generation microscopy, which monitored the native collagen fibers. Finally, a histopathological analysis of muscle tissues sections was carried out to monitor inflammation, necrosis and mean fiber diameter as a readout of myofiber maturity.

Specifically, sirius red staining of the tibialis anterior (TA) muscle demonstrated a 50% reduction in fibrillary collagen deposition in the LOX inhibited mice (FIGS. 6D_{iii,iv}, 6E, 6G). This staining further revealed that the connective tissue (primarily collagen fibers) was diffused in the control muscles. In contrast, the Fc-LPD treated ones were sharper and more distinct and the muscle was better organized as monitored by the circularity ratio (FIG. 6F). Histological evaluation of mdx mice tibialis anterior tissue showed reduction of inflammatory grade (˜25%) and fibrosis score (˜50%). Monitoring the assembly of fibrillary collagen in its native, unlabeled state by two-photon SHG microscopy, less collagen fibers signal with more linear direction was observed, after treatment with Fc-LPD. To quantify the changes in directional fiber orientation, Fourier component analysis for directionality on representative images obtained by SHG was applied (FIGS. 6D_{v, vi}, 6H). Moreover, two-photon SHG images showed thick fiber bundles in Fc-treated, which are absent in Fc-LPD-treated tissues. Altogether, these results demonstrate that treating the mice with this anti-LOX molecule inhibits fibrosis accumulation in a DMD background.

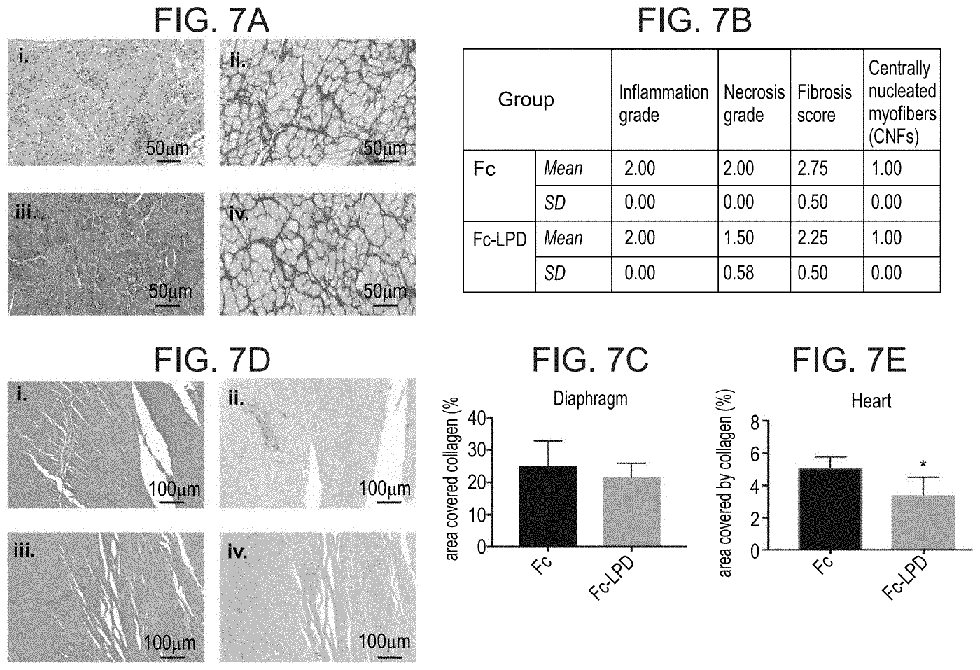

By examining the heart tissue, a significant ˜50% reduction in the amount of cardiac collagen deposition was observed in the Fc-LPD-treated mice when compared to those treated with Fc (FIGS. 7D, 7E). While at the commencement of the experiment (mdx mice at 6-7 months of age), cardiac and TA muscles were not highly fibrotic, the diaphragm was significantly affected hence it serves as a model for testing whether the anti-LOX treatment can promote the alleviation of an already fibrotic tissue. Sirius red staining of diaphragms of harvested mice, demonstrated a similar pattern to that observed in the cardiac and TA muscles in which a reduction in the amount of collagen deposition was observed (FIGS. 7A, 7C). Likewise, a pathologist report carried out suggested a ˜20% reduction in the fibrosis score (25% in Fc-treated vs. 21% in Fc-LPD-treated; (FIG. 7B).

### Example 5

### Analyzing the Significance of Glycosylation Sites to LPD Activity Cloning and Characterization

The pYD5-Fc-LPD plasmid was used as a template in order to create point mutations in the three N-glycosylated sites N81, 97, 144 in the LPD, which are the N346, 362, 409 in our Fc-LPD construct with Q SEQ ID NO: 11. The mutations were performed with site-directed mutagenesis according to kit manufacturer instructions. EC₅₀ of generated Fc-LPD N346, 362, 409Q inhibitor was measured by ELISA (FIG. 1C) ranged from 10 to 2000 nM against the LOX catalytic domain (EC₅₀=58 nmol/L).

Immunoprecipitation

Fc-LPD was coated on magnetic protein G beads (GE) according to manufacturer's instructions. The coated beads were incubated with concentrated supernatant from 10 cm² dish culture of HDF cells overnight on 4° C. Pellet beads were obtained by centrifuge (˜500 g for 5 min 4° C.) and the beads were washed with PBS of volume at least 5 times the initial beads volume. Then the beads pellet was placed in a collection column, which was inserted in an eppendorf tube containing 10 ul of 1M Tris-HCl pH 8. 90 ul of elution buffer (Thermo-Scientific) was added to the beads and after 5 min of incubation elution was performed by centrifuge. The last step was repeated four times.

Mass-spectrometry for identification gel bands from I.P of Fc-LPD The samples were eluted with 5% SDS and subjected to tryptic digestion using an S-trap. The resulting peptides were analyzed using nanoflow liquid chromatography (nanoAcquity) coupled to high resolution, high mass accuracy mass spectrometry (Q Exactive HFX). Each sample was processed with MaxQuant v1.6.0.16. The data was searched with the Andromeda search engine against the human proteome database appended with common lab protein contaminants and the following modifications: Carbamidomethylation of C as a fixed modification and oxidation of M and protein N-term acetylation as variable ones. The LFQ intensities (label free quantification) were calculated and used for further calculations using Perseus v1.6.07. Decoy hits were filtered out, as well as proteins that were identified on the basis of a modified peptide only. The LFQ intensities were log transformed and only proteins that had at least 2 valid values in at least one experimental group were kept. GO annotations were added.

Results

The Fc-LPD has three glycosylation sites, in order to investigate the importance of glycosylation a new construct Fc-LPD-N346, 362, 409Q was created. These two proteins including the Fc (as control) were used to clarify the interactions of LPD with ECM proteins in the supernatant of HDF cells, as in Example 3. For this purpose, HDF cells were cultured in 60 cm² dishes for 3 days in 10 ml serum-free and phenol red-free medium. The medium was collected and concentrated using 10 kDa cutoff vivaspin filters and incubated with Fc, Fc-LPD and Fc-LPD-N346, 362, 409Q, which were pre-incubated in magnetic protein G beads. Immunoprecipitation elutions were analysed by Mass Spectrometry and analysis of MS data showed the interactions of both Fc-LPD forms with ECM molecules, which are either part of core matrisome or matrisome-associated. Specifically, among the co-immunoprecipitated proteins were ECM glycoproteins (nidogen-1), secreted factors (filaggrin-2, hornerin), extracellular region or secreted proteins (HSP70, HSP90, HSP60). It is noteworthy to mention that the quantified ratio between Fc-LPD and Fc-LPD− N346, 362, 409Q was differentiated among the different proteins. For example, the ratio between non-glycosylated form and Fc control was significantly higher with Nidogen-1 and HSP70 compared to ratio of glycosylated Fc-LPD with Fc. (Table 2 below):

TABLE 2
Summary of proteins identified by mass spectrometry

| | | | | | Unique | | | | Ratio Fc- |
|---|---|---|---|---|---|---|---|---|---|
| | | | Molecular | No. of | sequence | | Ratio | Ratio Fc- | LPD:Fc- |
| | | | Mass | unique | coverage | | Fc- | LPD-N346, | LPDN346, |
| | UniProtKB | Name | (kDa) | peptides | [%] | Gene | LPD:Fc | 409, 456Q:Fc | 409, 456Q |
| ECM | P14543 | Nidogen-1 | 136.3 | 5 | 4.7 | NID1 | 9 | 13.7 | 0.7 |
| glycoproteins | | | | | | | | | |
| Secreted | Q5D862 | Filaggrin-2 | 247.9 | 2 | 2.6 | FLG2 | 1 | 0 | 55.7 |
| factors | Q86YZ3 | Homerin | 282.2 | 12 | 15.8 | HRNR | 1.3 | 0.2 | 6.7 |
| Extracellular | P17066/P48741 | Heat shock | 71 | 2 | 5.4 | HSPA6/7 | 78.4 | 194 | 0.4 |
| region or | | 70 kDa | | | | | | | |
| secreted | | protein 6/7 | | | | | | | |
| | P0DMV8/ | Heat shock | 70 | 22 | 41.3 | HSPA1A/B | 14.1 | 30 | 0.4 |
| | P0DMV9 | 70 kDa | | | | | | | |
| | | protein 1A/B | | | | | | | |
| | P07900 | Heat shock | 84.6 | 2 | 5.1 | HSP90AA1 | 0.7 | 0.2 | 2.9 |
| | | protein HSP | | | | | | | |
| | | 90-alpha | | | | | | | |
| | P11142 | Heat shock | 70 | 13 | 32.7 | HSPA8 | 1.2 | 0.9 | 1.4 |
| | | cognate 71 | | | | | | | |
| | | kDa protein | | | | | | | |
| | P08238 | Heat shock | 83 | 12 | 23.3 | HSP90AB1 | 1.2 | 1.8 | 0.6 |
| | | protein HSP | | | | | | | |
| | | 90-beta | | | | | | | |
| | P10809 | 60 kDa heat | 61 | 9 | 31.1 | HSPD1 | 1.8 | 0.1 | 6.5 |
| | | shock protein, | | | | | | | |
| | | mitochondrial | | | | | | | |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited in the Application

1. Lu S X, Holland A M, Na I K, Terwey T H, Alpdogan O, Bautista J L, Smith O M, Suh D, King C, Kochman A, Hubbard V M, Rao U K, Yim N, Liu C, Laga A C, Murphy G, Jenq R R, Zakrzewski J L, Penack O, Dykstra L, Bampoe K, Perez L, Furie B, Furie B, van den Brink M R (2010) Absence of P-selectin in recipients of allogeneic bone marrow transplantation ameliorates experimental graft-versus-host disease. J Immunol 185 (3): 1912-1919. doi:10.4049/jimmunol.0903148
2. Charge S B, Rudnicki M A (2004) Cellular and molecular regulation of muscle regeneration. Physiol Rev 84 (1): 209-238. doi:10.1152/physrev.00019.2003
3. Lo Cascio C M, Goetze O, Latshang T D, Bluemel S, Frauenfelder T, Bloch K E (2016) Gastrointestinal Dysfunction in Patients with Duchenne Muscular Dystrophy. PLoS One 11 (10):e0163779. doi:10.1371/journal.pone.0163779
4. Murphy M M, Lawson J A, Mathew S J, Hutcheson D A, Kardon G (2011) Satellite cells, connective tissue fibroblasts and their interactions are crucial for muscle regeneration. Development 138 (17): 3625-3637. doi: 10.1242/dev 0.064162
5. Burks T N, Cohn R D (2011) Role of TGF-beta signaling in inherited and acquired myopathies. Skelet Muscle 1 (1): 19. doi:10.1186/2044-5040-1-19
6. Serrano A L, Mann C J, Vidal B, Ardite E, Perdiguero E, Munoz-Canoves P (2011) Cellular and molecular mechanisms regulating fibrosis in skeletal muscle repair and disease. Curr Top Dev Biol 96:167-201. doi:10.1016/B978-0-12-385940-2.00007-3
7. Mendell J R, Rodino-Klapac L R (2016) Duchenne muscular dystrophy: CRISPR/Cas9 treatment. Cell Res 26 (5): 513-514. doi:10.1038/cr.2016.28
8. Meregalli M, Farini A, Belicchi M, Parolini D, Cassinelli L, Razini P, Sitzia C, Torrente Y (2013) Perspectives of stem cell therapy in Duchenne muscular dystrophy. FEBS J 280 (17): 4251-4262. doi:10.1111/febs.12083
9. Desguerre I, Arnold L, Vignaud A, Cuvellier S, Yacoub-Youssef H, Gherardi R K, Chelly J, Chretien F, Mounier R, Ferry A, Chazaud B (2012) A new model of experimental fibrosis in hindlimb skeletal muscle of adult mdx mouse mimicking muscular dystrophy. Muscle Nerve 45 (6): 803-814. doi:10.1002/mus.23341
10. Smith L R, Hammers D W, Sweeney H L, Barton E R (2016) Increased collagen cross-linking is a signature of dystrophin-deficient muscle. Muscle Nerve 54 (1): 71-78. doi: 10.1002/mus.24998
11. Lucero H A, Kagan H M (2006) Lysyl oxidase: an oxidative enzyme and effector of cell function. Cell Mol Life Sci 63 (19-20): 2304-2316. doi:10.1007/s00018-006-6149-9
12. Csiszar K (2001) Lysyl oxidases: a novel multifunctional amine oxidase family. Prog Nucleic Acid Res Mol Biol 70:1-32
13. Kagan H M, Li W (2003) Lysyl oxidase: properties, specificity, and biological roles inside and outside of the cell. J Cell Biochem 88 (4): 660-672. doi:10.1002/jcb.10413
14. Molnar J, Fong K S, He Q P, Hayashi K, Kim Y, Fong S F, Fogelgren B, Szauter K M, Mink M, Csiszar K (2003) Structural and functional diversity of lysyl oxidase and the LOX-like proteins. Biochim Biophys Acta 1647 (1-2): 220-224
15. Hornstra I K, Birge S, Starcher B, Bailey A J, Mecham R P, Shapiro S D (2003) Lysyl oxidase is required for vascular and diaphragmatic development in mice. J Biol Chem 278 (16): 14387-14393. doi:10.1074/jbc.M210144200
16. Maki J M, Rasanen J, Tikkanen H, Sormunen R, Makikallio K, Kivirikko K I, Soininen R (2002) Inactivation of the lysyl oxidase gene Lox leads to aortic aneurysms, cardiovascular dysfunction, and perinatal death in mice. Circulation 106 (19): 2503-2509
17. Maki J M, Sormunen R, Lippo S, Kaarteenaho-Wiik R, Soininen R, Myllyharju J (2005) Lysyl oxidase is essential for normal development and function of the respiratory system and for the integrity of elastic and collagen fibers in various tissues. Am J Pathol 167 (4): 927-936. doi:10.1016/S0002-9440(10)61183-2
18. Warburton D, Shi W (2005) Lo, and the niche is knit: lysyl oxidase activity and maintenance of lung, aorta, and skin integrity. Am J Pathol 167 (4): 921-922. doi:10.1016/S0002-9440(10)61181-9
19. Guo Y, Pischon N, Palamakumbura A H, Trackman P C (2007) Intracellular distribution of the lysyl oxidase propeptide in osteoblastic cells. Am J Physiol Cell Physiol 292 (6): C2095-2102. doi:10.1152/ajpcell.00613.2006
20. Li W, Nellaiappan K, Strassmaier T, Graham L, Thomas K M, Kagan H M (1997) Localization and activity of lysyl oxidase within nuclei of fibrogenic cells. Proc Natl Acad Sci USA 94 (24): 12817-12822
21. Payne S L, Fogelgren B, Hess A R, Seftor E A, Wiley E L, Fong S F, Csiszar K, Hendrix M J, Kirschmann D A (2005) Lysyl oxidase regulates breast cancer cell migration and adhesion through a hydrogen peroxide-mediated mechanism. Cancer Res 65 (24): 11429-11436. doi: 10.1158/0008-5472.CAN-05-1274
22. Giampuzzi M, Oleggini R, Di Donato A (2003) Altered adhesion features and signal transduction in NRK-49F cells transformed by down-regulation of lysyl oxidase. Biochim Biophys Acta 1647 (1-2): 239-244
23. Lazarus H M, Cruikshank W W, Narasimhan N, Kagan H M, Center D M (1995) Induction of human monocyte motility by lysyl oxidase. Matrix Biol 14 (9): 727-731
24. Li W, Liu G, Chou I N, Kagan H M (2000) Hydrogen peroxide-mediated, lysyl oxidase-dependent chemotaxis of vascular smooth muscle cells. J Cell Biochem 78 (4): 550-557
25. Nelson J M, Diegelmann R F, Cohen I K (1988) Effect of beta-aminopropionitrile and ascorbate on fibroblast migration. Proc Soc Exp Biol Med 188 (3): 346-352
26. Smith-Mungo L I, Kagan H M (1998) Lysyl oxidase: properties, regulation and multiple functions in biology. Matrix Biol 16 (7): 387-398
27. Kutchuk L, Laitala A, Soueid-Bomgarten S, Shentzer P, Rosendahl A H, Eilot S, Grossman M, Sagi I, Sormunen R, Myllyharju J, Maki J M, Hasson P (2015) Muscle composition is regulated by a Lox-TGFbeta feedback loop. Development 142 (5): 983-993. doi: 10.1242/dev 0.113449
28. Schindelin J, Arganda-Carreras I, Frise E, Kaynig V, Longair M, Pietzsch T, Preibisch S, Rueden C, Saalfeld S, Schmid B, Tinevez J Y, White D J, Hartenstein V, Eliceiri K, Tomancak P, Cardona A (2012) Fiji: an open-source platform for biological-image analysis. Nat Methods 9 (7): 676-682. doi:10.1038/nmeth.2019

What is claimed is:

1. A synthetic polypeptide comprising a propeptide of human lysyl oxidase (LOX), said polypeptide being devoid of LOX catalytic activity, said synthetic polypeptide comprising a modification which imparts said polypeptide with enhanced stability under physiological conditions as compared to a native form of said polypeptide not comprising said modification, wherein said modification comprises a proteinaceous modification and said polypeptide is a chimeric polypeptide.

2. The polypeptide of claim 1, wherein said proteinaceous modification is selected from the group consisting of immunoglobulin, human serum albumin, and transferrin.

3. The polypeptide of claim 2, wherein said immunoglobulin comprises an Fc domain.

4. A method of treating fibrosis in a subject in need thereof, the method comprising administering to the subject a polypeptide comprising a propeptide of lysyl oxidase (LOX), said polypeptide being devoid of LOX catalytic activity, wherein the method does not comprise administration of D-penicillamine, thereby treating fibrosis in the subject.

5. A method of treating DMD in a subject in need thereof, the method comprising administering to the subject a propeptide of lysyl oxidase (LOX), said polypeptide being devoid of LOX catalytic activity, thereby treating DMD in a subject in need thereof.

6. The polypeptide of claim 1, wherein said polypeptide is:
glycosylated on at least one or two or all of N81, N97 and N144 of SEQ ID NO: 1;
not glycosylated on N81, N97 and N144 of SEQ ID NO: 1;
characterized by an EC50 of 100-500 nM, as determined by an ELISA assay;
characterized by KD of 10-100 nM, as determined by a microscale thermophoresis;
characterized by a transition midpoint of 20-70° C., as determined by differential scanning fluorimetry (DSF); and/or
capable of reducing at least one of fibrillar collagen and cross-linked collagen, as determined by SHG microscopy.

7. The method of claim 4, wherein said fibrosis is not Ras-signaling dependent or not associated with cancer or a bone disease.

8. The polypeptide of claim 1, wherein said propeptide of lysyl oxidase (LOX) is of human LOX.

9. The method of claim 5, wherein the method does not comprise administration of D-penicillamine.

10. A method of inhibiting enzymatic activity of LOX, the method comprising contacting the LOX with the polypeptide of claim 1, thereby inhibiting the enzymatic activity of LOX.

11. The method of claim 10, performed in-vivo.

12. The method of claim 10, performed in-vitro.

13. The method of claim 10, performed ex-vivo.

14. A method of producing the polypeptide of claim 1, the method comprising expressing in a host cell, a polynucleotide encoding the polypeptide and when necessary, modifying the polypeptide with said chemical modification, thereby producing the polypeptide.

15. The method of claim 14, further comprising isolating the polypeptide from the host cell.

16. The polypeptide of claim 1, wherein the polypeptide is as set forth in SEQ ID NO: 1 or 5.

17. A polynucleotide encoding the polypeptide of claim 1.
